(19) United States
(12) Patent Application Publication
(10) Pub. No.: US 2009/0318548 A1
(43) Pub. Date: Dec. 24, 2009

(54) PARA-QUINOL DERIVATIVES AND METHODS OF STEREO SELECTIVELY SYNTHESIZING AND USING SAME

(75) Inventors: Guy L. Plourde, Prince George (CA); R. Randy Spaetzel, Prince George (CA)

Correspondence Address: OYEN, WIGGS, GREEN & MUTALA LLP;480 - THE STATION, 601 WEST CORDOVA STREET, VANCOUVER, BC, V6B 1G1, CA

(73) Assignee: UNIVERSITY OF NORTHERN BRITISH COLUMBIA, Prince George, BC (CA)

(21) Appl. No.: 12/438,926

(22) PCT Filed: Aug. 23, 2007
(86) PCT No.: PCT/CA2007/001494

§ 371 Date: Aug. 11, 2009

Related U.S. Application Data
(60) Provisional application No. 60/823,632, filed on Aug. 25, 2006.

(51) Int. Cl. A61K 31/343; C07D 307/94; C07D 493/20; C07D 497/10; A61K 31/34

(52) U.S. Cl. 514/462; 549/265

(57) ABSTRACT

This application relates to para-quinol derivatives, such as analogues of manumycins, aranorosins and gymnastatins. This application also relates to methods of synthesizing and using the para-quinol derivatives. In one embodiment of the invention a compound having the chemical structure (I) is provided wherein X₁ and X₂ are carbon atoms either joined by double bond or joined by a single bond and comprising constituents of an epoxide ring or a hydroxyethylene moiety; X₃ and X₄ are carbon atoms either joined by double bond or joined by a single bond and comprising constituents of an epoxide ring; R₁ is selected from the group consisting of branched alkyl chains, unbranched alkyl chains, cycloalkyl groups, aromatic groups, alcohols, ethers, amines, and substituted or unsubstituted ureas, esters, aldehydes and carboxylic acids; and R₂ is selected from the group consisting of H, OH and NHR₃ wherein R₃ is a nitrogen protecting group. In a particular embodiment of the invention R₁ is a polyunsaturated carbon chain as found in biologically active manumycins. The applicant's synthetic method may involve diasteroselective formation of a spirolactone in an oxidative spiroannulation process using tyrosine or a tyrosine derivative having a chiral centre as a starting material.

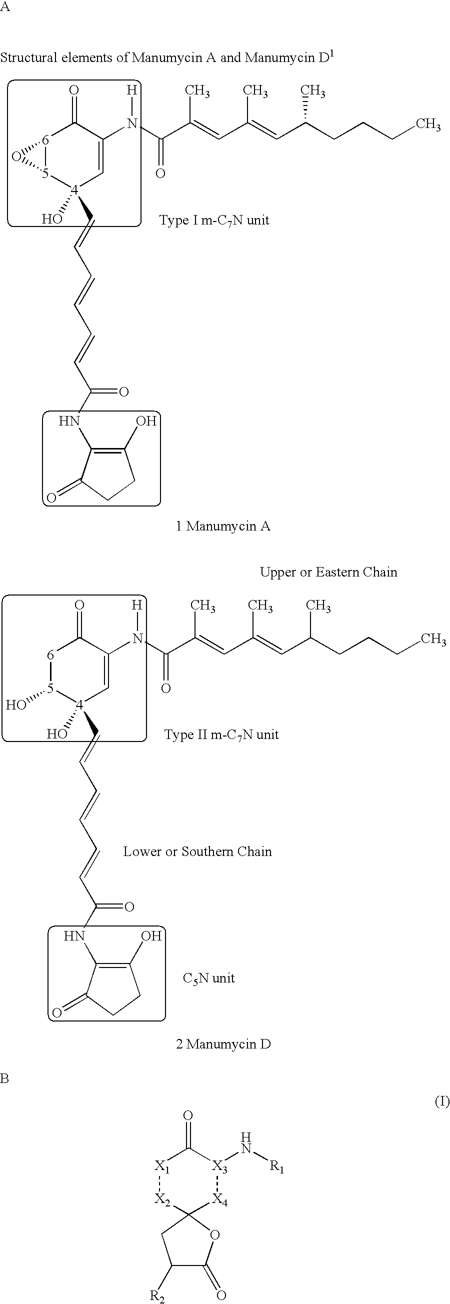

### REFERENCE TO RELATED APPLICATION

This application claims the benefit of U.S. provisional patent application Ser. No. 60/823,632 filed 25 Aug. 2006 which is hereby incorporated by reference in its entirety.

### TECHNICAL FIELD

This application relates to para-quinol derivatives, such as analogues of manumycins, aranorosins and gymnastatins. This application also relates to methods of synthesizing and using the para-quinol derivatives.

### BACKGROUND

The manumycin family of compounds are a class of secondary metabolites isolated from microbial origins that exhibit many interesting biological properties. For example, various manumycins have been shown to have antibiotic, antifungal, antiparasitic, anticoccidial, trypanocide, and insecticidal activities.¹ Manumycins may also be useful for inhibition of plants and animal enzymes. Numerous patents have issued for manumycin compounds such as U.S. Pat. No. 5,444,087, Patel et al., dated Aug. 22, 1995 which describes compounds derived from a strain of Streptomyces sp. having antibiotic and anti-tumour activity.

The manumycin class presently consists of 28 similarly structured molecules.^{2,3} The first member of the class, now referred to as Manumycin A, was isolated by Zähner and co-workers in 1963.² In 1973, Schröder and Zeeck proposed a novel structure for Manumycin A but the stereochemistry was later revised by Taylor and co-workers after they reported their synthesis of the enantiomer of the natural product.⁴ The common structural elements used to classify manumycins are: two unsaturated carbon chains, attached meta-fashioned to a distinctive functionalised cyclic core.⁶ The structural elements of Manumycin A 1 and Manumycin D 2 are shown in FIG. 1. The distinctive cyclic core of manumycins, often referred to as the m-C₇N unit, can exist as either a type I or a type II configuration. The type I configuration has an oxirane at the C-5/C-6 carbons of the cyclic core m-C₇N unit, while the type II configuration has a hydroxylethylene at the C-5/C-6 carbons of its m-C₇N unit, as illustrated in FIG. 1 with Manumycin A 1 (type I) and Manumycin D 2 (type II) respectively.^{3,5} Another common feature to many of the manumycins is a 2-amino-3-hydroxycyclopent-2-enone moiety (C₅N unit) linked to the “lower” or “southern” chain as shown in compound 2. While the family of manumycins retains many similar structural elements, the significant structural differences between manumycins occur mostly in the “upper” or “eastern” chain. These structural variations of the “eastern” chain involve different patterns of methyl branches and double bonds with varying lengths of polyunsaturated carbon chains attached to the m-C₇N unit.¹ The eastern side chains of manumycins are believed to be important for their biological activity. A list of naturally occurring manumycins showing the common cyclic core and the variable eastern chain is set forth in Appendix 1 attached hereto (the R₂ groups in Appendix 1 relate to the variable southern chain and pertain to that appendix only).

Due to the difficulty of producing cost effective quantities of manumycins from their bacterial source, various groups have developed methodologies for synthesizing some of the 28 naturally occurring manumycins. The total synthesis of a number of racemic and enantiopure manumycins have been accomplished by Taylor et al. They published the first manumycin to be synthesized, (+/−)-Alisamycin in 1996, using the methodology to make the M-C₇N unit derived from their earlier work with (+/−)-Bromoxone, and (+/−) LL-C10037α.⁷ Taylor and co-workers were soon to follow with the racemic synthesis of other manumycins: (+/−)-U-62162,⁹ (+/−)-Nisamycin¹⁰ and (+/−)-Colabomycin D.⁸ Wipf et al. also published a synthesis for (+/−)-Nisamycin in 1999, based on their methodology developed for the m-C₇N unit in their synthesis of (+/−) LL-C10037α.¹¹

In addition to manumycins, a number of smaller secondary metabolites displaying structural similarities to manumycins without the “eastern” and “southern” polyunsaturated chains have been isolated and characterized.^{12,13} Representative examples of these biologically active m-C₇N core analogues are found in FIG. 2 and include: MT 35214 3, LL-C10037α 4 Enaminomycin A 5, Enaminomycin B 6, Enaminomycin C₇, MM14201 8, Epoxydon 9¹, Epiepoxydon 10, Epoformin 11, Epiepoformin 12, Chaloxone 13, and Bromoxone 14.⁶⁴

The inventors have observed that the aranorosin and gymnastatin families of spirocompounds share many structural features with the central core m-C₇N unit of the manumycin family. This similarity is especially apparent if the spiroether/lactone moiety is imagined to be opened. Aranorosins and gymnastatins are cyclic spiro-tyrosine metabolites isolated from a number of different natural sources. Some examples of this class of compounds includes: Aranorosin 15, Aranosinol A 16 and B 17, Aranochlor A 18 and B 19 and Gymnastatins A 20, B 21, C 22, D 23, E 24 and I 25, as shown in FIGS. 3 and 4.

Aranorosin 15 was first isolated from the fermentation broth of Gymnascella dankaliensis (formerly named: Pseudoarachniotus roseus) and has been shown to exhibit positive biological activity towards a variety of fungi, bacteria and cancers on a micromolar scale in vitro.¹⁴ Aranorosin 15 has an unusual 1-oxaspiro[4.5]decane ring system (see FIG. 3); its total synthesis has been reported by the two research groups of Wipf^{15,16} and Taylor.¹⁷ Since its initial discovery in 1988, further Aranorosin like spiro-Tyrosine metabolites have been isolated and characterized. The spiro-tyrosine secondary metabolites Aranorosinol A 16 and B 17 were isolated in 1992 from a strain of Pseudoarachniotus roseus.¹⁸ Both compounds contain the 1-oxaspiro[4.5]decane ring system of Aranorosin 15 and exhibited in vitro inhibition of an assortment of bacteria and fungi in the μg mL⁻¹ range. A more recent examination of the biological properties of Aranorosinol A 16, showed the inhibition of POLO-like kinase 1 (Plk1) enzyme with a MIC of 118 μM.¹⁹ Plk1 is a highly conserved kinase enzyme that has been revealed to be over-expressed in cancer cell lines and has an essential role in cell regulation. Therefore, Plk1 is also a potential anti-cancer target for cancer research which is inhibited by a compound containing a 1-oxaspiro[4.5]decane ring system.

Another study looking for solutions to the rapid emergence of antibiotic resistance in pathogenic bacteria found and isolated Aranorosinol B 17 from a screening of 4000 microbes.²⁰ Aranorosinol B 17 is a potent inhibitor against autophosphorylation of YycG, an essential histidine kinase in the stress-response pathway in bacteria. Comparative experiments against the established antibiotics cefazolin, amikacin, vancomycin, erythromycin and ofloxacin determined negligible inhibition of Bacillus subtilis YycG whereas Aranorosinol B 17 inhibited YycG from both Bacillus subtilis and Staphylococcus aureus with an IC₅₀ of 223 and 211 μM respectively. Thus Aranorosinol B 17 inhibits a biological pathway in bacteria current antibiotics do not utilize and therefore could be developed to be used against antibiotic resistance in pathogenic bacteria.

Two new additions to the Aranorosin family of compounds include the isolation of secondary metabolites Aranochlor A 18 and B 19 in 1998 from Pseudoarachniotus roseus.²¹ Both metabolites contain the 1-oxaspiro[4.5]decane ring system of the other aranorosins and also inhibit a variety of bacteria and fungi in vitro in the mg mL⁻¹ range. What is new to the aranorosin type carbon skeletons of both aranochlors is the addition of a chloroalkene in place of one of the epoxides. With the incorporation of a vinyl halide functional group into their structure, Aranochlor A 18 and B 19 posses a strong structural similarity to the spirocyclized gymnastatins, a family of compounds with potent cytotoxicity towards various cancers.^{19,21}

The gymnastatins are cytotoxic metabolites isolated from a sponge-derived fungus Gymnascella dankaliensis.²² The fungus was discovered residing on the marine sponge Halichondria japonica which researchers were able to separate from the sponge and cultivate. After 4 weeks of growth in a salt water buffered medium, a variety of cytotoxic secondary metabolites were isolated from the fungus using multiple separation techniques to afford the following types of compounds: dankasterone, gymnasterones and gymnastatins.^{22,25} Most of the metabolites examined exhibited moderate to high biological activity towards cultured p388 lymphocytic leukemia with an ED₅₀ in the μg mL⁻¹ to ng mL⁻¹ range.

As shown in FIG. 4, the Gymnastatins A-E 20-24 and Gymnastatin I 25¹⁹ contain the 1-oxaspiro[4.5]decane ring system of the aranorosin family of compounds.²⁵ The total synthesis of Gymnastatin A 20 and Gymnastatin I 25, along with various analogues have been accomplished and have been used to establish the absolute stereochemistry of most of the compounds in this class.¹⁹ To date all the gymnastatins and aranorosins share the same 6R configuration at the 4,6-dimethyl-dodecadiene-2E,4E-oic acid unit attached to the nitrogen. The cytotoxic activity of Gymnastatins A-E 20-24 against cultured p388 lymphocytic leukemia was ED₅₀ 0.018 μg mL (20), 0.108 μg mL (21), 0.106 μg mL (22), 10.8 μg mL (23) and 10.8 μg mL (24) respectively.²⁵

It is apparent that analogues of naturally occurring manumycins, aranorosins and gymnastatins have considerable promise as drug candidates. However, in large measure existing synthetic techniques, which typically use benzoquinone methodology, lack stereoselective facial control. While some racemic manumycin benzoquinones exhibit similar biological activities as their natural product counterparts, there is an ongoing need for development of enantiopure compounds suitable for therapeutic purposes. In order to overcome the limitations of the prior art, the present invention relates to manumycin, aranorosin and gymnastatin analogues which may be diastereoselectively formed from para-quinols rather than benzoquinones. A comparison of the general structure of the benzoquinone (26) and para-quinol (27) analogues is set out in FIG. 5.

The para-quinol derivatives of the present invention more closely resemble the aranorosin and gymnastatin spirocompounds while maintaining a cyclic M-C₇N core and optional eastern side chain similar to the manumcycins.

### SUMMARY OF INVENTION

In accordance with the invention, a compound having the chemical formula formula (I) is provided:

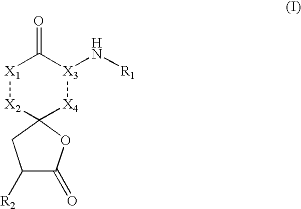

wherein X₁ and X₂ are carbon atoms either joined by double bond or joined by a single bond and comprising constituents of an epoxide ring or a hydroxyethylene moiety;
wherein X₃ and X₄ are carbon atoms either joined by double bond or joined by a single bond and comprising constituents of an epoxide ring;
wherein R₁ is selected from the group consisting of branched alkyl chains, unbranched alkyl chains, cycloalkyl groups, aromatic groups, alcohols, ethers, amines, and substituted or unsubstituted ureas, esters, aldehydes and carboxylic acids; and
wherein R₂ is selected from the group consisting of H, OH and NHR₃ wherein R₃ is a nitrogen protecting group.

In one particular embodiment R₁ may be a polyunsaturated carbon chain substituent as commonly found in naturally-occurring manumycin compounds, such as those listed in Appendix 1. In another particular embodiment the nitrogen protecting group may be a sulphonyl chain, such as a tosyl or nosyl group.

The invention also relates to a method of diasteroselective formation of a spirolactone comprising, in one embodiment, the steps of providing a starting material selected from the group consisting of tyrosine or a tyrosine derivative, wherein said starting material comprises a tethered chiral chain comprising an amino functional group; selectively protecting the amino functional group to produce an amino protected intermediate; and oxidatively spiroannulating the protected intermediate or a derivative thereof to preferentially form a diastereomer of the spirolactone.

In another embodiment the invention relates to a method of forming a para-quinol derivative including the steps of providing a phenol starting material comprising a nitro functional group; hydrogenating the starting material or a derivative thereof to transform the nitro group to an amine group and thereby produce an electron donating amine derivative; reacting the amine derivative with an acid chloride derivative to produce an amide derivative; and oxidatively spiroannulating the amide derivative to produce a spirolactone.

### BRIEF DESCRIPTION OF DRAWINGS

In drawings which are intended to illustrate the prior art and/or embodiments of the invention, but which should not be interpreted as restricting the spirit or scope of the invention in any way:

FIG. 1 illustrates the structural elements of Manumycin A and Manumycin D.

FIG. 2 shows the chemical formulas of biologically active m-C₇N core analogues.

FIG. 3 shows the chemical formulas of the aranorosin family of compounds.

FIG. 4 shows the chemical formulas of the gymnastatin family of spirocyclic compounds.

FIG. 5 shows a comparison between the general structure of the benzoquinone and para-quinol analogues of manumycins.

FIG. 6 shows the compound (+/−)-39 with the proposed relative stereochemistry and labeled carbons for the DEPT-135 NMR.

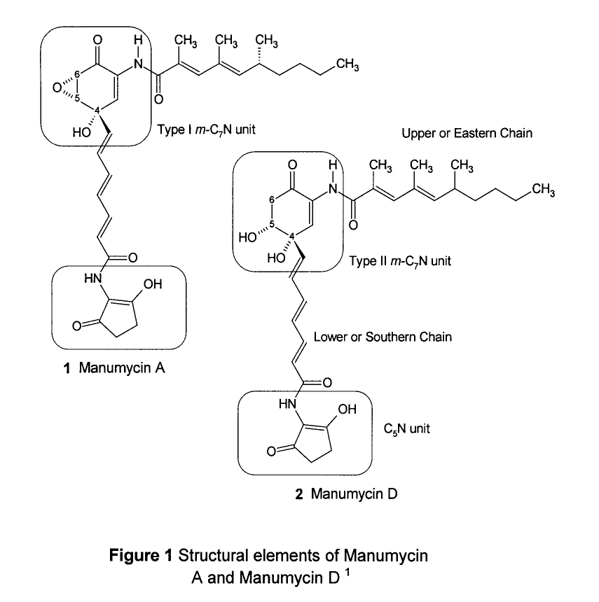

### DETAILED DESCRIPTION OF THE INVENTION

Throughout the following description, specific details are set forth in order to provide a more thorough understanding of the invention. However, the invention may be practiced without these particulars. In other instances, well known elements have not been shown or described in detail to avoid unnecessarily obscuring the invention. Accordingly, the specification and drawings are to be regarded in an illustrative, rather than a restrictive, sense.

Spirocompounds are a large class of inorganic and organic compounds that consist of multiple rings joined through a common carbon atom. A sub-category of this large class of compounds are cyclic spiro-tyrosine metabolites which may be isolated from a number of natural sources. As indicated above, aranorosins and gymnastatins are examples of this class of compounds. The present invention relates to para-quinol derivatives which are analogues of aranorosins and gymnastatins. The para-quinol derivatives may also include a cyclic core and functional groups which mimic the biologically active manumycin class of compounds.

As described below, the para-quinol derivatives of the present invention include spirolactones having the chemical structure (I);

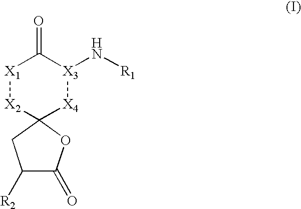

wherein X₁ and X₂ are carbon atoms either joined by double bond or joined by a single bond and comprising constituents of an epoxide ring or a hydroxyethylene moiety; X₃ and X₄ are carbon atoms either joined by double bond or joined by a single bond and comprising constituents of an epoxide ring; R₁ is selected from the group consisting of branched alkyl chains, unbranched alkyl chains, cycloalkyl groups, aromatic groups, alcohols, ethers, amines, and substituted or unsubstituted ureas, esters, aldehydes and carboxylic acids; and R₂ is selected from the group consisting of H OH and NHR₃ wherein R₃ is a nitrogen protecting group. The applicant's synthetic methods may involve diasteroselective formation of a spirolactone in an oxidative spiroannulation process using tyrosine or a tyrosine derivative having a chiral centre as a starting material.

The inventors initially selected an (L)-Tyrosine derivative as a suitable starting material for their asymmetric spiroannulation methodology. Full details of the methodology are set forth in the Examples below. The first step in the method development was to confirm that using a new substituent, Toluenesulfonyl (Ts), on the amino functional group of (L)-Tyrosine would not negatively affect the oxidative spiroannulation reaction.¹⁴ Typically, spiroannulation of Tyrosine has been done with other substituents (N-Ac, N-Cbz, N-BOC, N-phthalamido). Therefore, a number of test reactions using (L)-Tyrosine with a Ts residue attached to the amino functional group were carried out to evaluate whether the use of the new substituent was feasible. Additionally, these test reactions allowed the inventors to further optimize the conditions for the spiroannulation reaction having regard to the new type of starting materials.

It was determined that selective tosylation of only the amino functional group on the Tyrosine amino acid required the use of a protecting group. The innate nature of the Tyrosine compound is for tosylation to occur at both the amino and the phenolic hydroxyl functional groups concurrently. There are varying strategies in the literature involving multi-step synthesis with up to two different types of protecting groups for adding a substituent to the Tyrosine compound's amino functional group.^{26,27} However in 2001 Ciufolini et al., reported a three pot synthesis for the formation of (N-Ts)-Tyrosine 28^{28,29} but, in an effort to utilize an efficient total synthesis a novel two pot synthesis was developed to produce (N-Ts)-Tyrosine 28 (See Scheme 1). Instead of combating Tyrosine's propensity for quantitative di-tosylation with tosyl chloride,³⁰ the inventors alternatively cleaved the tosylate back into hydroxyl group forgoing the need for any additional reaction steps with protecting groups altogether.³¹ The different reaction conditions needed to cleave the tosyl group from the amino moiety³² on the (L)-Tyrosine compound led to simplification of the synthetic procedure for the formation of (N-Ts)-Tyrosine 28.

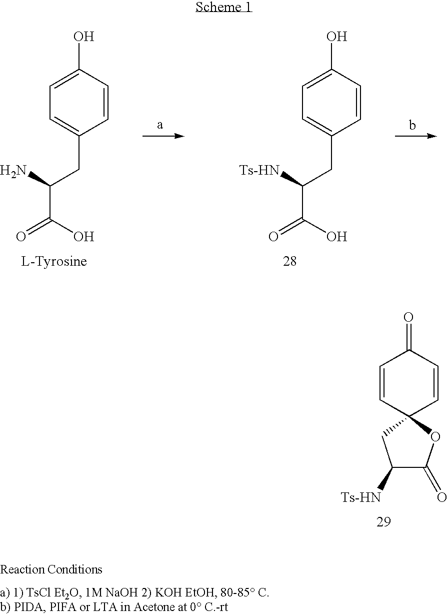

The di-tosylation of L-Tyrosine proceeded smoothly with the formation of a white suspension. The work up in the literature cited was temperamental³⁰ so it was modified to an acidification with hydrochloric acid (pH 1-2), followed by the extraction of the aqueous mixture with ethyl acetate to afford the crude intermediate di-tosyl-tyrosine. The crude product was then selectively detosylated with potassium hydroxide in ethanol overnight at 85-90° C. Compound 28 was obtained as a white solid in a 92% yield from the starting material (L)-Tyrosine after silica gel chromatography.

After the synthesis of compound 28, the oxidative spiroannulation reactions were evaluated using various reaction conditions with three different oxidants (Phenyliodine (III) diacetate (PIDA), Phenyliodine (III) bis(trifluoroacetate) (PIFA), Lead (IV) acetate (LTA))⁵⁵⁻⁵⁷ and were found to proceed to the (N-Ts)-Tyrosine spirolactone 29 with no unexpected complications. The PIFA in acetone reaction was repeated and purified for analytical information and afforded 29 in a 36% yield.

Since spirolactone 29 does not contain a new chiral centre at the spirocarbon, the use of 3-Nitro-Tyrosine as the starting material was investigated. It was determined from spiroannulation of a 3-Nitro-Tyrosine tosyl protected derivative that a tethered chiral chain could direct diastereoselective spirolactone formation. With reference to Scheme 2, formation of the (N-Ts)-3-Nitro-Tyrosine derivative 30 using the test reactions conditions from Scheme 1 occurred much more slowly and with a lower yield (25-54%). However, it was found that by substituting tetrahydrofuran (THF) for diethyl ether the reaction rate was increased along with the total isolated yield for compound 30 to 86%.

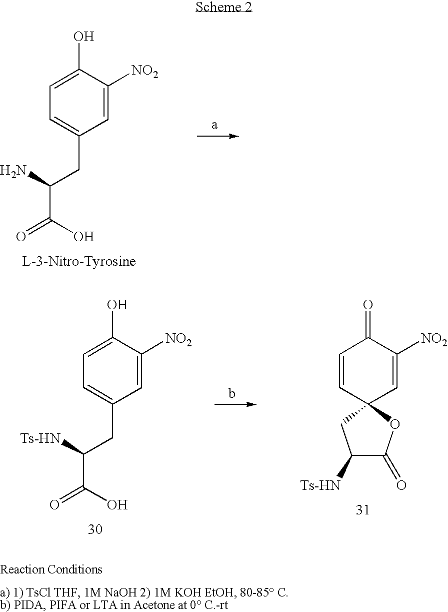

The inventors determined that the spiroannulation of compound 30, unlike the test reactions for the spiroannulation of compound 28, was difficult to achieve in the case of the 3-Nitro substitutent. It was decided that the nitro functional group being such a strong electron withdrawing group (EWG) at the 3-position on compound 30, might be interfering with the oxidative spiroannulation reaction.^{33,34} To rectify this problem, a new tyrosine derivative having an electron donating group (EDG) was developed.

To confirm the importance of an EDG, test reactions were carried out as shown in Scheme 3 using a simplified non-Tyrosine version of (N-Ts)-3-Nitro-Tyrosine 30 but bearing an EDG instead of an EWG.

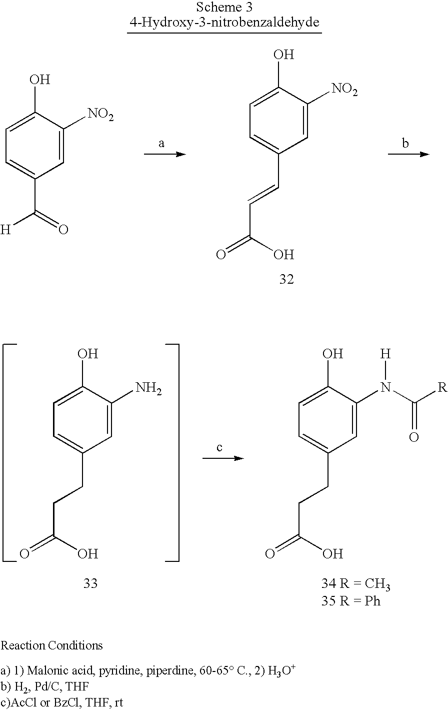

To make the starting material, a Knoevenagel condensation with the Doebner modification was used to extend the aldehyde on 4-Hydroxy-3-nitrobenzaldehyde to a propenoic acid moiety giving 32 in 93% yield. Compound 32 could be used without purification.^{35,36} Hydrogenation transformed the nitro functional group to an amine while also reducing the alkene on the para-chain to a propanoic acid residue.³⁷ Since compound 33 decomposes quickly with handling, the amine was directly converted into two different types of amides with either acetyl chloride or benzoyl chloride, making the new EDG's at the 3-position of the benzene ring for compounds 34 and 35 with yields of 48% and 83% from 32.³⁸

The inventors next confirmed that oxidative spiroannulation of EDG derivatives 34 and 35 could be successfully achieved using the same reaction conditions as in the test reaction described above (1 to 3 equivalents oxidant, in acetone at 0° C.). As shown in Scheme 4, oxidative spiroannulation proceeded with good yields for both compounds using all three oxidants as determined by 1H-NMR spectra of the crude products.

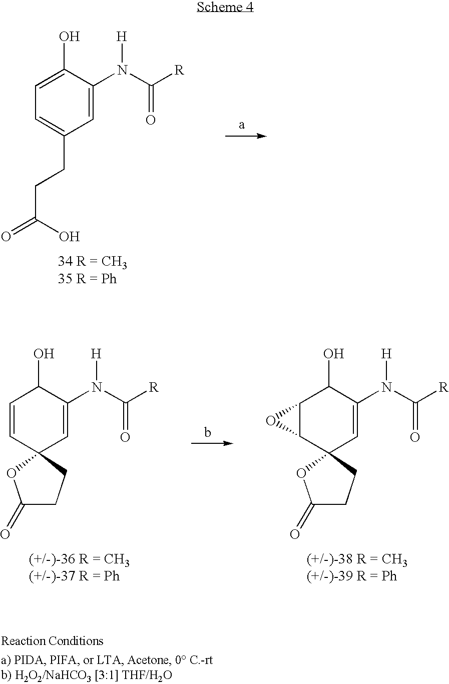

The reactions with 34 and 35 using PIFA in acetone were repeated on a larger scale and purified giving 96% and 86% yields for (+/−)-36 and (+/−)-37 respectively.

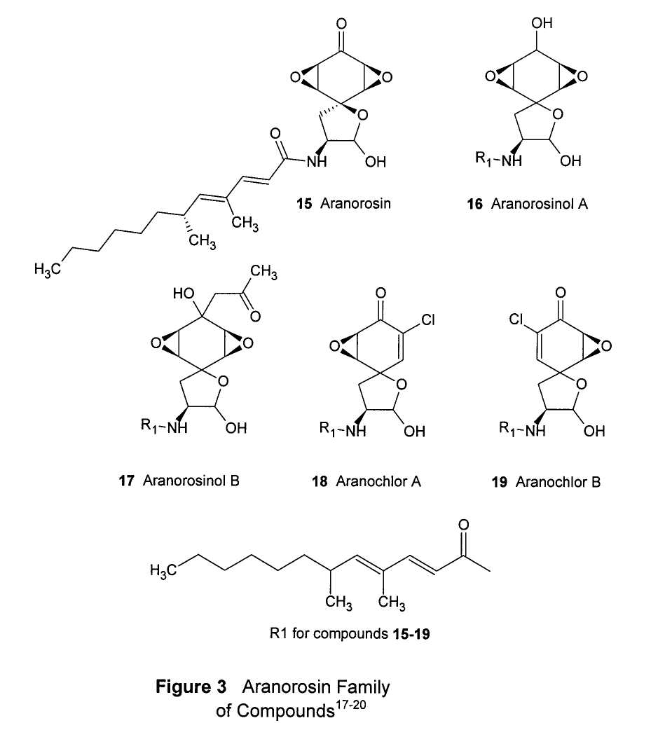

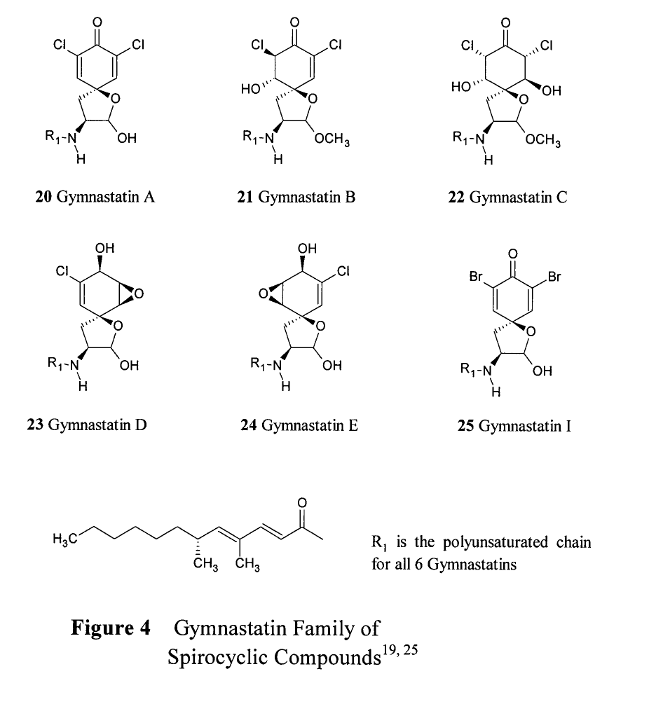

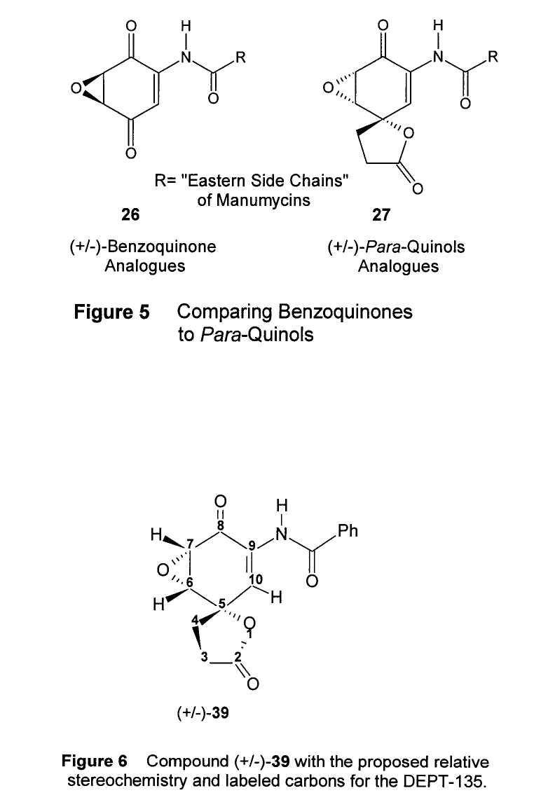

The amide spirolactones (+/−)-36 and (+/−)-37 both contain a racemic mixture of enantiomers and share a spirolactone carbon skeleton with the Aranorosin (see FIG. 3) and Gymnastatin (see FIG. 4) families of compounds. Furthermore, as will be appreciated by a person skilled in the art, the electron poor alkene (or least substituted alkene functional group) of compound 36 and 37 may be selectively epoxidized in accordance with methods well known in the literature, the teachings of which are hereby incorporated by reference: Magdziak et al.,³⁹ Marco-Contelles et al.,⁴⁰ Runcie and Taylor,⁴¹ Matsumoto et al.,⁴² Barros et al.,⁴³ Barrett et al.,⁴⁴ Nicolaou et al.,⁴⁵ Alcaraz et al.,⁴⁶ and Suzuki et al.⁴⁷ Selective epoxidation reactions are known in the prior art for preferentially attacking either an electron rich double bond or an electron poor double bond, or both double bonds of a spirocompound. For example, Magdziak et al., Cyclohexadienone Ketals and Quinols: Four Building Blocks Potentially Useful for Enantioselective Synthesis, Chem. Rev. 2004, 104, 1383-1429,³⁹ incorporated herein by reference, describe selective expoxidation reactions for preferentially attacking a single double bond or both double bonds of a spirocompound (at pages 1409 and 1410). By way of specific example, the following expoxidation reaction conditions could be selected:

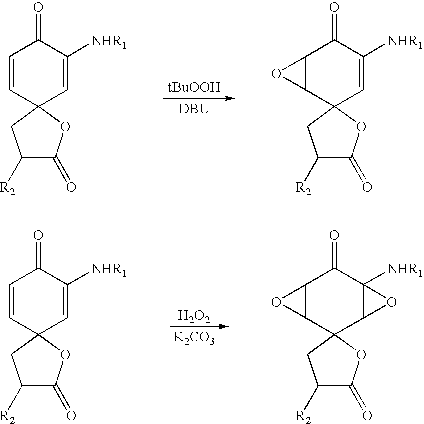

Hydrogen peroxide reactions on the racemic mixture of compound 37 were examined to confirm that it was possible to control the regio and stereoselective epoxidation of spirocompounds 36 and 37. The un-optimized treatment of 37 with hydrogen peroxide and sodium bicarbonate in THF and water, led to the epoxidation of the electron poor alkene on 37 giving compound (+/−)-39 in a low isolated yield of 7%.⁵⁸⁻⁶³ The regioselective aspect of the epoxidation on the electron poor alkene was confirmed by a Distortionless Enhancement by Polarization Transfer—135 (DEPT-135) NMR spectrum. The two carbons of the epoxide ring with one attached hydrogen each (a methine group, (CH)), showed two CH signals in the DEPT-135 NMR spectrum at 51.5 ppm (C-6) and 55.4 ppm (C-7). These signals were in the range one would expect to see epoxide CH signals (40-80 ppm from the internal standard of tetramethylsilane (TMS)). Conversely, if the epoxide had formed on the electron rich alkene (or more substituted alkene) there would have been only one CH signal, as C-9 is a quaternary carbon and does not have hydrogen attached. The stereoselective aspect of the syn epoxidation, between the oxirane and the lactone's oxygen, also has literature precedent for nucleophilic reactions occurring with high 7′-facial selectivity towards the alkene on the cyclohexadienone ring and to proceed with the syn configuration with respect to the lactone oxygen.^{14, 48-51} Hence it is believed epoxidation will produce (+/−)-39 with the relative stereochemistry as shown in FIG. 6.

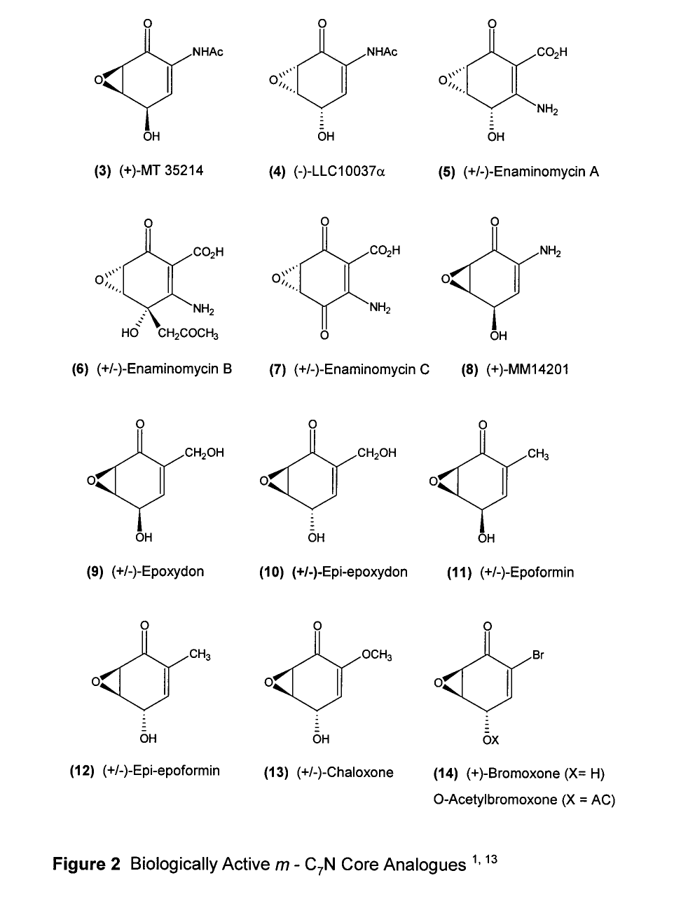

With reference to FIG. 2, novel compounds 38 and 39 including an epoxide ring represent racemic analogues of the m-C₇N unit's carbon skeleton found in the manumycin family. As will be appreciated by a person skilled in the art, the synthetic methodology illustrated in Schemes 3 and 4 is a convenient means to attach alternative carbon side chains (i.e. substituent R₁ in structure (I) above) to the amine group on the cyclic core. As described above, the amines are treated with an acid chloride to prepare the corresponding amides. A list of possible R₁ side chains which are present in naturally occurring manumycins is set forth in Appendix 1 (the R₂ groups in Appendix 1 relate to the variable southern chain and pertain to that appendix only). As mentioned above, it is believed that such “eastern” side chains of manumycins are important for their biological activity.

The use of EDG intermediates has also been successfully employed to achieve diastereoselective synthesis of spirolactones. As shown in Scheme 5, diastereoselective formation of the spirolactones using a tethered chiral chain was accomplished using the EDG Tyrosine derivates 41 to make the spirolactone compounds (+)-42 and 43.

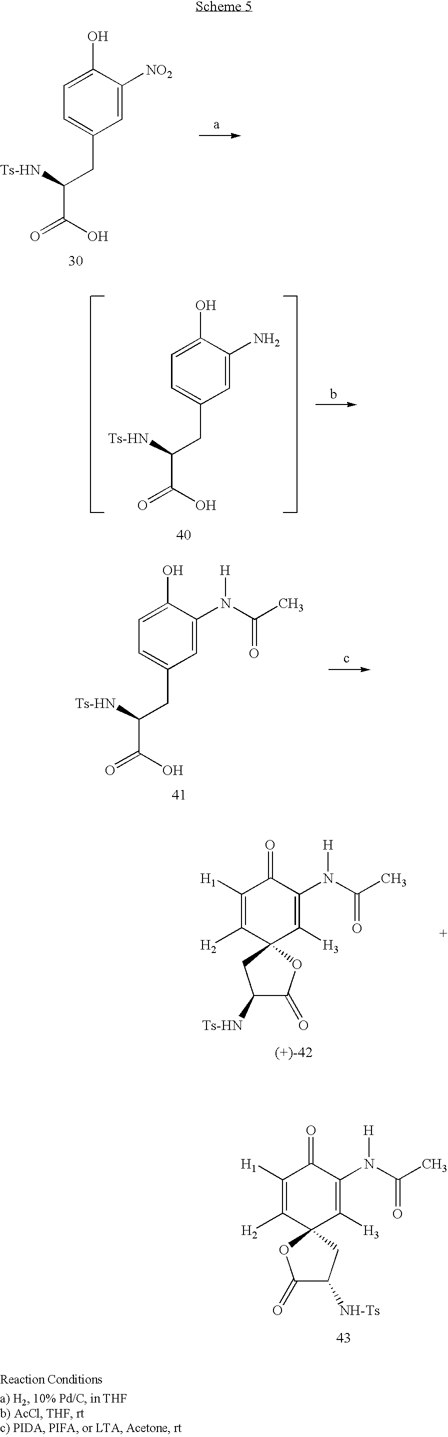

The previously prepared compound 30 (Scheme 2) was reduced to the amine 40 using hydrogen with a 10% palladium/carbon catalyst. Due to instability problems with purifying the crude product, compound 40 was reacted directly with AcCl to form the amide 41 in a 59% yield for the two reactions. With an EDG at the 3-position on the benzene ring of the Tyrosine derivative, the oxidative spiroannulation reactions with the three oxidants proceeded as expected using the methodology described above (1 to 3 equivalents oxidant, in acetone at 0° C.). As described with reference to the Examples below, it was clear from ¹H-NMR spectra that the crude products were a mixture of diastereomers. The use of an EDG on the 3-position of the aromatic ring for compound 41 enabled the spiroannulation reactions to proceed successfully and allowed the formation of the diastereomers with selectivity. Using the integration of the ¹H-NMR spectrum signals for H1, H2, and H₃ of the crude products 42 and 43 (Scheme 5) the ratio of diastereomers was estimated. The ¹H-NMR spectrum peaks' integration ratios for H₁ (6.25 ppm major-6.30 ppm minor diastereomer), H₂ (7.06 ppm major-6.92 ppm minor diastereomer), and H₃ (7.52 ppm major-7.66 ppm minor diastereomer) were compared to calculate a major to minor ratio of [3:1] for compound (+)-42 and 43 respectively. After separating the two diastereomers (+)-42 and 43 by silica gel chromatography, a higher ratio of major and minor diastereomers (using their isolated masses, 18 mg: 1 mg) was obtained. A larger scale reaction using PIFA as the oxidant, gave a silica gel chromatography separation of 38 mg for the major diastereomer, 54 mg for a mixture of major and minor diastereomers, and only 8 mg of the minor diastereomer (total yield of 85%).

Scheme 6 shows a proposed reaction mechanism for the PIFA oxidant reacting with compound 41 in order to prepare the two diastereomers as described above.

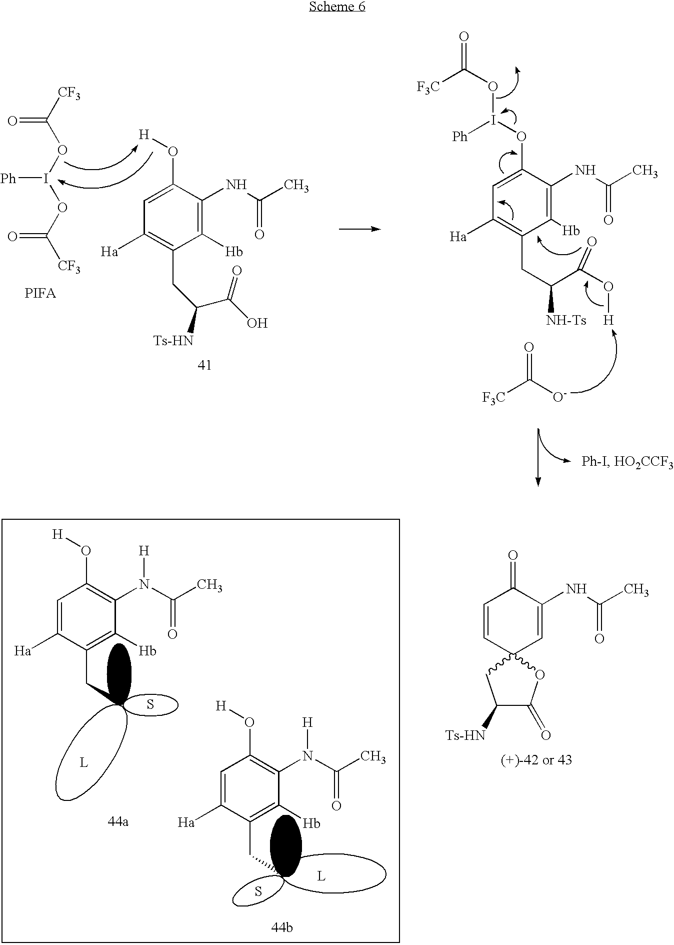

PIFA reacts with the phenolic hydroxyl creating a hypervalent iodine complex. The conjugate base deprotonated the carboxylic acid which initiated the spiral annulation of the hypervalent iodine complex creating the two diastereomers. The structure 44 in Scheme 6 represents the anticipated diastereomeric transition state where the labels S, M and L represent the functional groups and their size: S for hydrogen, M for carboxylic acid and L for toluene sulfonylamide. The proximity of the tethered chiral chain to the aromatic ring limits the free rotation of the chiral center and the chiral center's interaction with the hydrogen protons Ha and Hb on the aromatic ring causes the resulting 3 to 1 diastereoselectivity. According to this proposed reaction mechanism the transition state of 44a leads to the major isomer (+)-42. In this configuration the large sulfonylamide group orients itself on the side of the proton Ha where steric factors are less important than the transition state 44b. In the transition state 44b a model study shows a larger steric hindrance between the sulfonylamide, the proton Hb, and the acetamide functional groups, thus leading to the minor diastereomer 43.

### EXAMPLES

The following examples are intended to illustrate embodiments of the invention in further detail and are not intended to be construed in a limiting manner.

### Example 1

### Materials and Methods

Infrared (IR) spectra were recorded on a FT-IR Perkins System 2000 spectrophotometer. Mass spectra were recorded with a Hewlett Packard (Agilent) 5989 B Mass spectrometer (MS) with a 5890 Series II Gas Chromatograph (GC). Optical rotations were obtained using a Rudolf Research Autopol III instrument. Flash column chromatographies were carried out using Silicycle silica gel (230-400 mesh, 60 Å). Analytical thin layer chromatography (tlc) was carried out on silica gel coated aluminum plates from Silicycle (60 Å, indicator F-254, thickness 250 μm). Visualization of tlc-plates was accomplished with UV light (Short-wave UV, 254 nm) and/or by staining with Vanillin (27 g of vanillin, 50 mL water, 380 mL ethanol, 20 mL conc. sulfuric acid). ¹H (300.13 MHz) and ¹³C (75.47 MHz) NMR spectra were recorded on a Bruker AMX 2-300 spectrometer using tetramethylsilane (TMS) as an internal calibration standard when using deuterated chloroform. When using other deuterated solvents, spectra were calibrated using chemical shifts of residue protons of the deuterated solvent used. Chemical shifts (δ) are quoted in parts per million (ppm) and ¹H spin coupling (J) values are in Hz. Two-dimensional NMR spectra were used to elicit further chemical shift information and confirm compound structures.

### General Methodology of Small Scale Oxidations:

PIDA: To the starting material (20-30 mg, 1 eq.) dissolved in acetone (10 mL, 0° C.) was added PIDA in one portion (2.1 eq.) and the solution was stirred until completion. Reaction progress was followed by tlc (40-60 min). The solution was diluted with ethyl acetate (20-25 mL) and washed with cold water (10 mL). The organic fraction was dried (MgSO₄) and the solvent was evaporated. The residue was left under vacuum overnight to evaporate off phenyl iodine.

PIFA: To the starting material (20-30 mg, 1 eq.) dissolved in acetone (10 mL, 0° C.) was added PIFA in one portion (1.01 eq.) and the solution was stirred until completion. Reaction progress was followed by tlc (15-30 min.). The solution was diluted with ethyl acetate (20-25 mL) and washed with cold water (10 mL). The organic fraction was dried (MgSO₄) and the solvent was evaporated. The residue was left under vacuum overnight to evaporate off phenyl iodine.

LTA: To the starting material (20-30 mg, 1 eq.) dissolved in acetone (10 mL, 0° C.) was added LTA in one portion (3 eq.) and the solution was stirred until completion. Reaction progress was followed by tlc (15-30 min.). Ethylene glycol (4-5 drops) was added to the solution and it was left to stir overnight (14-16 hrs). The reaction mixture was filtered through Celite® while rinsing with acetone (10-20 mL) and the solvent was evaporated. The residue was left under vacuum overnight to evaporate off ethylene glycol.

(2S)-2-{([(4-methlphenyl)sulfonyl]amino}-3-(4-{[(4-methylphenyl)sulfonyl]oxy}phenyl)propanoic acid Intermediate to 28: To a solution of L-Tyrosine (374 mg, 2.06 mmol, 1 eq.) in 1M NaOH (50 mL) was added a solution of TsCl (2.843 g, 14.91 mmol, 7 eq.) in diethyl ether (100 mL) in three portions (5 min. apart) at room temperature and the resulting mixture was stirred vigorously for 4-5 hrs. (confirmed by tlc: [2:8] MeOH/CHCl₃). The resulting white suspension was acidified with 10% HCl (pH 1) and extracted with ethyl acetate (3×100 mL). The organic fractions were combined, washed with saturated NaCl (150 mL) and dried (MgSO₄). The solvent was then evaporated to afford a white solid (crude 938 mg, 93% yield). The product was used in the following reaction without further purification. Molecular Formula—C₂₃H₂₃NO₇S₂. Formula Weight—489.56 g mole⁻¹. R_{f} (EtOAc)=0.65. ¹H-NMR (CDCl₃) δ: 2.40 (s, 3H, H-7′), 2.44 (s, 3H, H-7″), 2.90 (m, 1H, H-3a), 3.08 (m, 1H, H-3b), 4.12 (m, 1H, H-2), 5.28 (d, 1H, J=8.8, N—H), 6.81 (d, 2H, J=8.3, H-5, H-9), 6.99 (d, 2H, J=8.3, H-6, H-8) 7.21 (d, 2H, J=8.1, H-2′, H-6′), 7.31 (d, 2H, J=8.1, 11-2″, H-6″), 7.55 (d, 2H, J=8.1, H-3′, H-5′), 7.67 (d, J=8.1, H-3″, H-5″) 9.28 (broad s, 1H, CO₂H). ¹³C-NMR (CDCl₃) δ: 21.74 (C-7″), 21.94 (C-7′), 38.24 (C-3), 56.49 (C-2), 122.69 (C-5, C-9), 127.20 (C-3′, C-5′) 128.66 (C-3″, C-5″), 129.96 (C-2′, C-6′), 130.04 (C-2″, C-6″) 130.87 (C-6, C-8), 132.31 (C-4), 134.17 (C-4′), 136.31 (C-4″), 144.32 (C-1′), 145.75 (C-1″), 148.99 (C-7), 175.30 (C-1).

(2S)-3-(4-hydroxylphenyl)-2-{[(4-methylphenyl)sulfonyl]amino}propanoic acid (28): To a solution of the intermediate of 28 (750 mg, 1.534 mmol, 1 eq.) in ethanol (100 mL) was added a solution of 1M 1KOH (50 mL) and the white suspension was heated (77-82° C.) while stirring for 6-7 hrs (confirmed by tlc: [1:1] EtOAc/Hexane after mini work up, 10% HCl and extracting with EtOAc.). The resulting mixture was left to cool, then acidified with 10% HCl (pH ˜1) and extracted with ethyl acetate (150 mL, 50 mL). The organic fractions were combined, washed with saturated NaCl (150 mL) and dried (MgSO₄). The solvent was then evaporated to afford a white solid. This crude product was purified by column chromatography on silica gel, eluting with 40% ethyl acetate/hexane to afford an off white solid (477 mg, 92% yield isolated). Molecular Formula—C₁₆H₁₇NO₅S. Formula Weight—335.38 g moles⁻¹. R_{f} (EtOAc)=0.10. ¹H-NMR (CD₃CN) δ: 2.39 (s, 3H, H-7′), 2.74 (m, 1H, H-3a), 2.92 (m, 1H, H-3b), 3.96 (m, 1H, H-2), 5.88 (d, 1H, J=8.8, N—H), 6.63 (d, 2H, J=8.4, H-5, H-9), 6.92 (d, 2H, J=8.4, H-6, H-8) 7.25 (d, 2H, J=8.1, H-2′, H-6′), 7.52 (d, 2H, J=8.3, H-3′, H-5′). ¹³C-NMR (CD₃CN) δ: 21.59 (C-7′), 38.52 (C-3), 58.27 (C-2), 116.06 (C-5, C-9), 127.77 (C-3′, C-5′), 128.20 (C-4), 130.54 (C-2′, C-6′), 131.52 (C-6, C-8), 138.47 (C-4′), 144.58 (C-1′), 156.90 (C-7), 172.71 (C-1).

N-[(3S)-1-oxaspiro[4.5]deca-6,9-dien-2,8-dion-3-yl]-4-methylbenzene sulfonamide (29): To a solution of 28 (177 mg, 0.528 mmol, 1 eq.) dissolved in acetone (15 mL, 0° C.) was added PIFA (229 mg, 0.533 mmol, 1.01 eq.) in one portion and the resulting mixture was stirred for 45-50 minutes (confirmed by tlc: [1:1] EtOAc/Hexane). The mixture was diluted with ethyl acetate (50 mL), then washed with cold water. The organic fraction was dried (MgSO₄) and the solvent evaporated to afford a Tan solid. The crude product was purified by column chromatography on silica gel, eluting with 50% ethyl acetate/hexane to afford an off white solid (63 mg, 36% yield isolated). Molecular Formula—C₁₆H₁₅NO₅S. Formula Weight—333.359 g mole⁻¹. ¹H-NMR (CDCl₃) δ: 2.48 (s, 3H, H-7′), 4.40 (m, 1H, H-2), 5.91 (d, 1H, J=5.8, N—H), 6.26 (dd, 2H, J=2, 10, H-5, H-9), 6.80 (dd, 2H, J=2, 10, H-6, H-8), 7.33 (d, 2H, J=8, H-3′, H-5′), 7.80 (d, 2H, J=8.2, H-2′, H-6′)

(2S)-3-(4-hydroxyl-3-nitrophenyl)-2-{[(4-methylphenyl)sulfonyl]amino}propanoic acid 30: To a solution of 3-Nitro-L-Tyrosine (1.029 g, 4.55 mmol, 1 eq.) dissolved in 1M NaOH (100 mL) was added a solution of tetrahydrofuran (150 mL) with TsCl (7.012 g, 36.9 mmol, 9.5 eq.) in three portions (5-10 min. apart) and the resulting orange solution was stirred vigorously at room temperature. After 25-30 min. the solution turned a yellow colour, indicating an acidic environment (pH ˜3), therefore more 1M NaOH (25 mL) was added. The solution then returned to an orange colour, which was left to stir overnight (14-16 hrs, confirmed by tlc ([2:8] MeOH/CHCl₃). The reaction mixture was acidified with 10% HCl (pH 1-2, orange to yellow colour change) and extracted with dichloromethane (100 mL, 50 mL). The organic fractions were combined, dried (MgSO₄) and the solvent evaporated to afford a yellow solid.

The crude yellow product was then dissolved in ethanol (100 mL) and 1M KOH (50 mL) was added. The now orange reaction mixture was warmed (80-85° C.) and left to stir overnight (12-14 hrs. confirmed by tlc: [2:8] MeOH/CHCl₃, after mini work up, 10% HCl and EtOAc). The resulting reaction mixture was cooled and then acidified with 10% HCl (pH 1-2) causing the orange solution to turn yellow. Then the reaction mixture was concentrated and yellow precipitates formed which were then extracted with dichloromethane (2×150 mL). The organic fractions were combined, dried (MgSO₄), and the solvent was then evaporated leaving a yellow solid. Recrystallization in benzene and drying under vacuum afforded a yellow solid product (1.485 g, 86% yield). Molecular Formula—C₁₆H₁₆N₂O₇S. Formula Weight—380.374 g mole⁻¹. Mpt: 137° C. [α]_{D}=−58.1⁰ (c: 0.155 g 100 mL⁻¹ at 21° C.). FT-IR (KBR disk) cm⁻¹: 1734 (CO₂H), 1326, 1158 (SO₂NHR), 1539, 1430 (NO₂). ¹H-NMR (CDCl₃) δ: 2.40 (s, 3H, H-7′), 2.91 (m, 1H, H-3a), 3.15 (m, 1H, H-3b), 3.74 (broad s, 1H, OH), 4.16 (m, 1H, H-2), 5.42 (d, 1H, J=8.5, N—H), 7.01 (d, 1H, J=8.6, H-8), 7.20 (d, 2H, J=8.3, H-2′, H-6′), 7.38 (dd, 1H, J=2.2, 8.6, H-9), 7.56 (d, 2H, J=8.3, H-3′, H-5′), 7.74 (d, 1H, J=2.2, H-5), 10.46 (broad s, 1H, CO₂H). ¹³C-NMR (CDCl₃) δ: 21.74 (C-7′), 37.70 (C-3), 56.59 (C-2), 120.41 (C-8), 125.59 (C-5), 127.18 (C-3′, C-5′), 127.73 (C-4), 129.91 (C-2′, C-6′), 133.32 (C-4′), 136.36 (C-7), 139.15 (C-9), 144.39 (C-1′), 154.50 (C-6), 174.65 (C-1).

(2E)-3-(4-hydroxyl-3-nitrophenyl)acrylic acid 32: To a solution of 4-hydroxyl-3-nitrobenzaldehyde (1.073 g, 6.43 mmol, 1 eq.) dissolved in pyridine (25 mL) was added piperidine (25 drops) and the resulting mixture was stirred (4-5 min.). Malonic acid (1.671 g, 16.1 mmol, 2.5 eq.) was then added in one portion and the resulting mixture was warmed (60-63° C.) and stirred overnight (12-14 hrs, confirmed by tlc: EtOAc, mini work up, 10% HCl and EtOAc). The reaction was cooled and acidified (50% HCl) until yellow precipitate formed (pH ˜2). This yellow precipitate was extracted with ethyl acetate (2×150 mL). The organic fractions were combined and washed with brine (150 mL), dried (MgSO₄), and the solvent was evaporated to afford a yellow solid. Removed excess solvent by vacuum and used without further purification (1.250 g, 93% yield). Molecular Formula—C₉H₇NO₅. Formula Weight—209.156 g mole⁻¹. FT-IR (KBR disk) cm⁻¹: 2942 (OH), 1684 (CO₂H), 1626 (C═C), 1533, 1270 (NO₂). ¹H-NMR (Acetone-D₆) δ: 2.87 (broad s, 1H, OH), 6.58 (d, 1H, J=16.0, H-2), 7.27 (d, 1H, J=8.8, H-8), 7.70 (d, 1H, J=16.4, H-3), 8.08 (d, 1H, J=2.2, 8.5, H-9), 8.40 (d, 1H, J=2.2, H-5), 10.67 (broad s, 1H, CO₂H). The ¹³C-NMR of this compound agrees with the previously published data.⁵²

3-(3-amino-4-hydroxylphenyl)propanoic acid 33: Detail procedure for formation of 33 can be found in experimental procedure of 34 and 35 below. Initial attempts to purify by recrystallization and column chromatography failed due to reactive qualities of product therefore used filtered solution of 33 directly for producing both amides 34 and 35. A small aliquot was used to acquire a ¹H-NMR of the crude product. Molecular Formula—C₉H₁₁NO₃. Formula Weight—181.189 g mole⁻¹. ¹H-NMR (D₃C-OD) δ: 2.46 (t, 2H, J=7.0, H-3), 2.69 (t, 2H, J=7.9, H-2), 6.48 (m, 1H, H-5), 6.60 (m, 2H, H-8, H-9),

3-[3-(acetylamino)-4-hydroxylphenyl]propanoic acid 34: To a solution of 32 (210 mg, 1.00 mmol, 1 eq.) dissolved in THF (20 mL) was added the catalyst 10% palladium-on-charcoal (15% by mass, 32 mg). The resulting mixture was then placed on a hydrogenator, flushed (5 times) with hydrogen, and left to agitate under pressure (39 psi.) for 6-7 hrs. The reaction mixture was vented and then vacuumed filtered through Celite® rinsing with THF (25-30 mL). AcCl (79 mg, 1.13 mmol, 1.13 eq.) was directly added to the filtered solution containing 33 and left to stir at room temperature for 60 min. Water was added (15 mL) and extracted with EtOAc (2×50 mL). The organic fractions were combined and washed with saturated NaCl (50 mL), dried (MgSO₄), and the solvent was evaporated off. The product was re-crystallized with Hexane/Acetone to afford a white solid (104 mg), with a 47% yield from compound 32. Molecular Formula—C₁₁H₁₃NO₄. Formula Weight—223.225 g mole⁻¹. FT-IR (KBR disk) cm⁻¹: 3393 (NH, OH), 1699 (CO₂H), 1657 (NHAc). ¹H-NMR (CD₃CN) δ: 2.15 (s, 3H, H-2′), 2.54 (t, 2H, J=7.5, H-3), 2.78 (t, 2H, J=7.5, H-2), 6.82 (d, 1H, J=8.3, 1H-8), 6.95 (dd, 1H, J=2.1, 8.3, H-9), 7.06 (d, 1H, J=2.0, H-5), 8.53 (broad s, 1H, OH), 8.81 (s, 1H, NH). ¹³C-NMR (CD₃CN) δ: 23.53 (C-2′), 30.96 (C-3), 35.96 (C-2), 119.50 (C-5), 123.04 (C-8), 127.10 (C-6), 127.31 (C-9), 133.69 (C-4), 147.89 (C-7), 172.09 (C-1), 174.42 (C-1′).

3-[3-(benzoylamino)-4-hydroxylphenyl]propanoic acid 35: To a solution of 32 (222 mg, 1.06 mmol, 1 eq.) dissolved in THF (20 mL) was added the catalyst 10% palladium-on-charcoal (15% by mass, 33 mg). The resulting mixture was then placed on a hydrogenator, flushed (5 times) with hydrogen and left to agitate under pressure (36 psi.) overnight (12 hrs) while recharging hydrogen pressure twice (36 psi.) until hydrogen up-take by reaction mixture stopped (pressure did not decrease for 1-2 hrs.). The reaction mixture was vacuum filtered through Celite® rinsing with THF. To the filtered solution containing 33 was directly added BzCl (154 mg, 1.1 mmol, 1 eq.) and left to stir at room temperature for 30 min. Then 10% HCl (25 mL) was added and stirring continued an additional 5 min. followed by extraction with CH₂Cl₂ (2×35 mL). The organic fractions were combined, dried (MgSO₄), and evaporated off solvent. The resulting mixture was re-crystallized with Hexane/Acetone to afford an off white solid (250 mg) with an 83% yield from compound 32. Molecular Formula —C₁₆H₁₅NO₄. Formula Weight—285.295 g mole⁻¹. FT-IR (KBR disk) cm⁻¹: 3201 (NH, OH), 1692 (CO₂H), 1636 (NHAc). ¹H-NMR (Acetone-D₆) δ: 2.60 (t, 2H, J=7.4, H-3), 2.84 (t, 2H, J=7.9, H-2), 6.89 (d, 1H, J=8.2, H-8), 7.00 (dd, 1H, J=2.1, 8.25, H-9), 7.57 (m, 4H, H-5, H-4′, H-5′, H-6′), 8.05 (d, 2H, J=8.2, H-3′, H-7′), 9.07 (broad s, 1H, NH), 9.54 (broad s, 1H, OH), 10.58 (broad s, 1H, CO₂H). ¹³C-NMR (Acetone-D₆) δ: 30.87 (C-3), 36.21 (C-2), 118.69 (C-8), 123.31 (C-5), 123.41 (C-6), 126.88 (C-9), 127.37 (C-4), 128.54 (C-4′, C-6′), 129.61 (C-3′, C-7′), 132.99 (C-5′), 134.99 (C-2′), 148.03 (C-7), 167.34 (C-1′), 173.94 (C-1).

N-(1-oxaspiro[4.5]deca-6,9-dien-2,8-dion-7-yl)acetamide (+/−)-36: To a solution of 34 (122 mg, 0.547 mmol, 1 eq.) dissolved in acetone (10 mL, 0° C.) was added PIFA (306 mg, 0.711 mmol, 1.3 eq.) in one portion and stirred for 20-25 minutes (confirmed by tlc: [1:1] EtOAc/Hexane). The reaction mixture was diluted with ethyl acetate (15 mL), washed with cold water (10 mL), dried organic fraction (MgSO₄) and evaporated off solvent to afford a Tan solid. The crude product was purified by re-dissolving with CHCl₃, filtering of the solution through Celite®, evaporating off the solvent and placing it under vacuum overnight to afford an off white solid (120 mg, 98% yield). Molecular Formula—C₁₁H₁₁NO₄. Formula Weight—221.209 g mole¹. FT-IR (KBR disk) cm⁻¹: 3333 (NH), 1777 (lactone), 1668 (amide), 1650 (ketone), 1620 (α, β-conjugation to ketone). ¹H-NMR (CDCl₃) δ: 2.17 (s, 3H, H-2′), 2.44 (m, 2H, H-4), 2.81 (m, 2H, H-3), 6.35 (d, 1H, J=10.0, H-9), 6.94 (dd, 1H, J=3.1, 10.0, H-10), 7.75 (d, 1H, J=3.1, H-6), 7.99 (broad s, 1H, NH). ¹³C-NMR (CDCl₃) δ: 24.86 (C-2′), 28.36 (C-4), 32.91 (C-3), 79.76 (C-5), 124.30 (C-6), 127.12 (C-9), 131.55 (C-7), 148.37 (C-10), 169.51 (C-1′), 175.46 (C-2), 179.40 (C-8).

N-(1-oxaspiro[4.5]deca-6,9-dien-2,8-dion-7-yl)benzamide (+/−)-37: To a solution of 34 (262 mg, 0.92 mmol, 1 eq.) dissolved in acetone (30 mL, 0° C.) was added PIFA (396 mg, 0.92 mmol, 1 eq.) in one portion and stirred for 20-25 minutes (confirmed by tlc: [1:1] EtOAc/Hexane). The reaction mixture was diluted with ethyl acetate (100 mL), washed with cold water (50 mL), dried organic fraction (MgSO₄) and the solvent was evaporated to afford a Tan solid. The crude product was purified by column chromatography on silica gel, eluting with (40) % ethyl acetate/hexane to afford an off white solid (243 mg, 86% yield isolated). Molecular Formula—C₁₆H₁₃NO₄. Formula Weight—283.279 g mole¹. FT-IR (KBR disk) cm⁻¹: 3381 (NH), 1781 (lactone), 1665 (amide), 1650 (ketone), 1621 (α, β-conjugation to ketone). ¹H-NMR (CDCl₃) δ: 2.49 (m, 2H, H-4), 2.84 (m, 2H, H-3), 6.42 (d, 1H, J=10.0, H-9), 6.99 (dd, 1H, J=3.1, 10.0, H-10), 7.54 (m, 3H, H-4′, H-5′, H-6′), 7.86 (m, 2H, H-3′, H-7′), 7.95 (d, 1H, J=3.1, H-6), 8.81 (broad s, 1H, NH). ¹³C-NMR (CDCl₃) δ: 28.40 (C-4), 33.02 (C-3), 79.84 (C-5), 124.54 (C-6), 127.20 (C-9), 127.32 (C-4′, C-6′), 129.16 (C-3′, C-7′), 131.74 (C-7), 132.72 (C-5′), 133.87 (C-2′), 148.60 (C-10), 166.21 (C-1′), 175.44 (C-2).

N-[(1′S,2R,6′R)-5,5′-dioxo-4,5-dihydro-3H-spiro furan-2,2′-[7]oxabicyclo[4.1.0]hept[3] en]-4′-yl]benzamide 39: To a solution of (+/−)-37 (55 mg, 0.14 mmol, 1 eq.) dissolved in 3:1 THF/H₂O (4 mL, 0° C.) was added ˜30% H₂O₂ (200 μL, 67 mg, 2 mmol, 14 eq.) and stirred (6 hrs, confirmed by tlc: [1:1] EtOAc/Hexane). The reaction mixture was diluted with water (15 mL) and extracted with ethyl acetate (2×25 mL). The organic fractions were combined, dried (MgSO₄), and evaporated solvent to afford a solid. The crude product was purified by column chromatography on silica gel, eluting with 50% ethyl acetate/hexane to afford an off white solid. The resulting mixture was triturated with HPLC hexane to remove some impurity (3 mg, 7% yield). Molecular Formula—C₁₆H₁₃NO₅. Formula Weight—299.278 g mole⁻¹. ¹H-NMR (CDCl₃) δ: 2.48 (t, 2H, J=8.4, H-3), 2.85 (m, 2H, H-4), 3.72 (d, 1H, J=4, H-7), 3.77 (m, 1H, H-6), 7.54 (m, 3H, H-4′, H-5′, H-6′), 7.72 (d, 1H, J=2.7, H-10), 8.35 (broad s, 1H, NH). ¹³C-NMR (CDCl₃) δ: 27.92 (C-4), 33.02 (C-3), 51.47 (C-6), 55.37 (C-7), 81.54 (C-5), 123.89 (C-9), 127.26 (C-4′, C-6′), 129.01 (C-3′, C-7′), 129.20 (C-10), 132.81 (C-5′), 133.75 (C-2′), 166.28 (C-1′), 174.86 (C-2), 188.18 (C-8).

(2S)-3-(3-amino-4-hydroxylphenyl)-2-{[(4-methylphenyl)sulfonyl]amino}propanoic acid 40: Initial attempts to purify by recrystallization and column chromatography failed due to reactive qualities of product therefore used filtered solution directly for producing the amide 41. A small reaction was repeated to acquire a ¹H-NMR for analytical information of the crude product (see experimental procedure of 41 below). Molecular Formula —C₁₆H₁₈N₂O₅S. Formula Weight—350.391 g mole⁻¹. ¹H-NMR (D₂O+Na₂CO₃) with a trace of EtOH solvent, δ: 2.35 (s, 3H, H-7′), 2.45 (m, 1H, H-3a), 2.78 (m, 1H, H-3b), 3.59 (m, 1H, H-2, EtOH), 6.33 (m, 3H, H-5, H-8, H-9), 7.22 (d, 2H, J=8.6, H-3′, H-5′), 7.39 (d, 2H, J=8.3, H-2′, H-6′).

(2S)-3-[3(acetylamino)-4-hydroxylphenyl]-2-{[(4-methylphenyl)sulfonyl]amino}propanoic acid 41: To a solution of 30 (166 mg, 0.437 mmol, 1 eq.) dissolved in THF (25 mL) was added the catalyst 10% palladium-on-charcoal (15% by mass, 26 mg). The resulting mixture was then placed on a hydrogenator, flushed (5 times) with hydrogen and left to agitate under pressure (39 psi.) for 15-16 hrs. The reaction mixture was vented and then vacuumed filtered through Celite® rinsing with THF (25-30 mL). To the filtered solution containing 40 was directly added AcCl (51 mg, 0.66 mmol, 1.5 eq.) and left to stir at room temperature overnight. The reaction mixture was diluted with CH₂Cl₂ (50 mL), washed with saturated NaCl (15 mL), dried (MgSO₄) and evaporated off solvent. It was then re-crystallized with Hexane/Acetone to afford a white solid (68 mg), with a 40% yield from compound 30. Molecular Formula—C₁₈H₂₀N₂O₆S. Formula Weight—392.427 g mole⁻¹. [α]_{D}=−88.9° (c: 0.018 g 100 mL⁻¹ at 21° C.). FT-IR (KBR disk) cm⁻¹: 3257 (NH), 1777 (CO₂H), 1657 (amide), 1289, 1157 (SO₂). ¹H-NMR (Acetone-D₆) and a trace of EtOAc solvent, δ: 2.20 (s, 3H, H-2″), 2.38 (s, 3H, H-7′), 2.83 (m, 1H, H-3a), 2.93 (m, 1H, H-3b), 4.04 (m, 1H, H-2, EtOAc), 6.64 (d, 1H, NH-2), 6.70 (d, 1H, J=8.2, H-8), 6.83 (dd, 1H, J=2.1, 8.2, H-9), 7.15 (broad s, 1H, H-5), 7.25 (d, 211, J=7.9, H-2′, H-6′), 7.55 (d, 2H, J=8.3, H-3′, H-5′), 9.25 (broad s, 1H, CO₂H). ¹³C-NMR (Acetone-D₆) δ: 21.48 (C-2″), 23.50 (C-7′), 37.70 (C-3), 58.29 (C-2), 118.97 (C-5), 123.04 (C-8), 127.30 (C-6), 127.73 (C-3′, C-5′), 127.87 (C-9), 128.72 (C-4′), 130.25 (C-4, C-2′, C-6′), 139.11 (C-1″), 143.80 (C-1′), 148.34 (C-7), 172.61 (C-1).

N-((3S)-3-{[(4-methylphenyl)sulfonyl]amino}-1-oxaspiro[4.5]deca-6,9-dion-2,8-dien-7-yl)acetamide (+)-42: To a solution of 41 (118 mg, 0.30 mmol, 1 eq.) dissolved in acetone (20 mL, 0° C.) was added PIFA (142 mg, 0.331 mmol, 1.1 eq.) in one portion. After confirming reaction completion by tlc (25 min.), the reaction mixture was diluted with EtOAc (25 mL), washed with cold water (15 mL), dried solvent (MgSO₄) and evaporated off solvent. The crude product was purified by column chromatography on silica gel, eluting with 50% ethyl acetate/hexane to afford the major and minor diastereomer as white solids. The resulting mixture was triturated with HPLC hexane to remove most of the impurity. Isolated 38 mg of the major diastereomer, 54 mg of a mixture of the major and minor diastereomer and 8 mg of the minor diastereomer (100 mg total, 85% total yield). Analytical information is reported for major diastereomer plus 42. Molecular Formula—C₁₈H₁₈N₂O₆S. Formula Weight—390.410 g mole⁻¹. [α]_{D}=+55.6° (c: 0.036 g 100 mL⁻¹ at 22° C.). FT-IR (KBR disk) cm⁻¹: 3327 (NH), 1778 (lactone), 1654 (amide), 1645 (ketone), 1631 (α, β-conjugation to ketone), 1339, 1161 (SO₂). ¹H-NMR (CD₃CN) trace of acetone solvent, δ: 2.16 (s, 3H, H-7′), 2.28 (m, 1H, H-4a), 2.45 (s, 3H, H-2″), 2.46 (m, 1H, H-4b), 3.54 (m, 1H, H-3), 6.12 (d, 1H, J=8.0, NH-2), 6.25 (d, 1H, J=10.0, H-9), 7.06 (dd, 1H, J=3.1, 10.0, H-10), 7.38 (d, 2H, J=8,0, H-2′, H-6′), 7.52 (d, 1H, J=3.1, H-6), 7.78 (d, 2H, J=8.3, H-3′, H-5′), 8.18 (broad s, 1H, NH-7). ¹³C-NMR (CD₃CN) δ: 21.59 (C-2″), 24.72 (C-7′), 39.96 (C-4), 52.97 (C-3), 78.42 (C-5), 124.78 (C-6), 127.48 (C-9), 127.95 (C-3′, C-5′), 130.84 (C-2′, C-6′), 133.51 (C-7), 138.81 (C-4′), 145.16 (C—I′), 147.41 (C-10), 170.93 (C-1″), 173.63 (C-2).

NMR spectra of compounds 28-30, 33-37, and 39-42 are set forth in the attached Appendix 2 in numerical order (all values are in parts per million (ppm)).

As will be apparent to those skilled in the art in the light of the foregoing disclosure, many alterations and modifications are possible in the practice of the invention without departing from the spirit or scope thereof. Accordingly, the scope of the invention is to be construed in accordance with the substance defined by the following claims.

### REFERENCES

1) Sattler, I.; Thiericke, R.; Zeeck, A. The Manumycin-group metabolites. Nat. Prod. Rep. 1998, 221-240.
2) Buzzetti F.; Gäumann E.; Hütter R.; Keller-Scheirlein W.; Neipp L.; Prelog V. and Zähner H. Stoffwechselprodukte von Mikroorganismen. Pharm. Acta Helv., 1963, 38, 871.
3) Hu, Y.; Floss, H. G. New Type II Manumycins Produced by Streptomyces nodosus ssp. Asukaensis and their biosynthesis. J. Antibiot. 2001, 54, 340-348.
4) Alcaraz, L.; Macdonald, G.; Ragot, J. P.; Lewis, N.; Taylor, R. J. K. Manumycin A: Synthesis of the (+)-Enantiomer and Revision of Sterochemical Assignment. J. Org. Chem. 1998, 63, 3526-3527.
5) Kohno J.; Nishio M.; Kawano K.; Nakanishi N.; Suzuki S.; Uchida T.; Komatsubara S.; TMC-1 A, B, C, and D, new antibiotics of the manumycin group produced by Stertomyces sp. Taxonomy, production, isolation, physico-chemical properties, structure elucidation and biological properties. J. Antibiot., 1996, 49, 1212.
6) Zeek A.; Schroeder K.; Frobel K; Grotte R. and Theiricke R. The structure of manumycin I. Characterization, structure and elucidation and biological activity, J Antibiot., 1987, 40, 1530.
7) Alcaraz, L.; Macdonald, G.; Kapfer, I.; Lewis, N. J.; Taylor, R. J. K. The first total synthesis of a member of the manumycin family of antibiotics: Alisamycin. Tet. Lett. 1996, 37, 6619-6622.
8) Wei, X.; Cronje Grove, J. J.; Taylor, R. J. K. The first total synthesis of (+/−)-colabomycin D. J. Chem. Soc., Perkin Trans. 1, 1999, 1143-1145.
9) Alcaraz, L.; Taylor, R. J. K. The first synthesis of the Streptomyces derived antibiotic U-62162. Chem. Commun. 1998, 1157-1158.
10) Taylor, R. J. K.; Alcaraz, L.; Kapfer-Eyer, I.; Macdonald, G.; Wei, X.; Lewis, N. The synthesis of alisamycin, nisamycin, LL-C10037α and novel epoxyquinol and epoxyquinone analogues of manumycin A. Synthesis, 1998, 775-790.
11) Wipf, P.; Coish, P. D. G. Total synthesis of (+/−)-Nisamycin. J. Org. Chem. 1999, 64, 5053-5061.
12) Edwards, R. L.; Maitland, D. J.; Scowen, I. J.; De Sousa, A. J. T.; Whalley, A. J. S. Metabolites of the higher fungi. Part 32. Rosnecatrone, a phytotoxic bicycle[4.1.0]hept-3-en-2-one from the fungus Rosellinia necatrix Prill. J. Chem. Soc., Perkin Trans 1. 2001, 537-542.
13) Marco-Contelles, J.; Molina, M. T.; Anjum, S. Naturally occurring cyclohexane epoxides: Sources, biological activities and synthesis. Chem. Rev. 2002, 1-44.
14) Rama Rao, A. V.; Gurjar, M. K.; Sharma, P. A. Studies directed towards the total synthesis of aranorosin. Tet. Lett. 1991, 32, 6613-6616.
15) Wipf, P.; Kim, Y. Stereoselective synthesis of the functionalized spirocyclic core of aranorosin. J. Org. Chem. 1993, 58, 1649-1650.
16) Wipf, P.; Kim, Y.; Fritch, P. C. Total synthesis and structure assignment of the antitumor antibiotic aranorosin. J. Org. Chem. 1993, 58, 7195-7203.
17) McKillop, A.; McLaren, L.; Taylor, R. J. K.; Watson, R. J.; Lewis, N. J. The total synthesis of the diepoxycyclohexanone antibiotic aranorosin and novel synthetic analogues. J. Chem. Soc., Perkin Trans. 1 1996, 1385-1393.
18) Roy, K,; Vijayakumar, E. K. S.; Mukhopadhyay, T.; Chatterjee, S.; Bhat, R. G.; Blumbach, J.; Ganguli, B. N. Aranorosinol A and aranorosinol B, two new metabolites from Pseudoarachniotus roseus: Production, isolation, structure elucidation and biological properties. J. Antibiot. 1992, 45, 1592-1598.
19) Phoon, C. W.; Somanadhan, B.; Heng, S. C. H.; Ngo, A.; Ng, S. B.; Butler, M. S.; Buss, A. D.; Sim, M. M. Isolation and total synthesis of gymnastatin N, a POLO-like kinase 1 active constituent from the fungus Arachniotus punctatus. Tetrahedron 2004, 60, 11619-11628.
20) Watanabe, T.; Hashimoto, Y.; Yamamoto, K.; Hirao, K.; Ishihama, A.; Hino, M.; Utsumi, R. Isolation and characterization of inhibitors of the essential histidine kinase, YycG in Bacillus subtilis and Staphylococcus aureus. J. Antibiot. 2003, 56, 1045-1052.

21) Mukhopadhyay, T.; Bhat, R. G.; Roy, R.; Yijayakumar, E. K. S.; Ganguli, B. N. Aranochlor A and aranochlor B, two new metabolites from Pseudoarachniotus roseus: Production, isolation, structure elucidation and biological properties. J. Antiot. 1997, 51, 439-441.
22) Amagata, T.; Doi, M.; Tohgo, M.; Minoura, K.; Numata, A. Dankasterone, a new class of cytotoxic steroid produced by a Gymnascella species from a marine sponge. Chem. Commun. 1999, 1321-1322.
23) Numata, A.; Amagata, T.; Minoura, K.; Ito, T. Gymnastatins, Novel cytotoxic metabolites produced by a fungal strain from a sponge. Tet. Lett. 1997, 38, 5675-5678.
24) Amagata, T.; Minoura, K.; Numata, A. Gymnasterones, Novel cytotoxic metabolites produced by a fungal strain from a sponge. Tetrahedron Letters, 1998, 39, 3773-3774.
25) Amagata, T.; Doi, M.; Ohta, T.; Minoura, K.; Numata, A. Absolute stereostructures of novel cytotoxic metabolites, gymnastatins A-E, from a Gymnascella species separated from a Halichondria sponge. J. Chem. Soc., Perkin Trans. 1 1998, 3585-3599.
26) Yamashiro, D.; Li, C. H. Adrenocorticotropins. 44. Total synthesis of the human hormone by the solid-phase method. J. Am. Chem. Soc. 1973, 95, 1310-1315.
27) Nicolaou, K. C.; Boddy, C. N. C.; Li, H.; Koumbis, A. E.; Hughes, R.; Natarajan, S.; Jain, N. F.; Ramanjulu, J. M.; Brase, S.; Solomon, M. E. Total Synthesis of Vancomycin—Part 2: Retrosynthetic Analysis, Synthesis of Amino Acid Building Blocks and Strategy Evaluations. Chem. Eur. J. 1999, 5, 2602-2621.
28) Ousmer, M.; Braun, N. A.; Bavoux, C.; Perrin, M.; Ciufolini, M. A. Total Synthesis of Tricyclic Azaspirane Derivatives of Tyrosine: FR901483 and TAN1251C. J. Am. Chem. Soc. 2001, 123, 7534-7538.
29) Ousmer, M.; Braun, N. A.; Ciufolini, M. A. Total Synthesis of FR901483. Org. Lett. 2001, 3, 765-767.
30) Furniss, B. S.; Hannaford, A. J.; Smith, P. W. G.; Tatchell, A. R. Vogel's: Textbook of Practical Organic Chemistry, 5^{th} Edition, John Wiley & Sons, Inc., New York, N.Y., 1989, p. 1280.
31) Wolfrom, M. L.; Koos, E. W.; Bhat, H. B. Osage Orange Pigments. XVIII. Synthesis of Osajaxanthone. J. Org. Chem. 1967, 32, 1058-1060.
32) Greene, T. W.; Wuts, P. G. M. Protective Groups in Organic Synthesis, 3^{rd} Edition, John Wiley & Sons Inc., Canada, 1999, pp. 604, 744-5.
33) Akiba, K.; Kobayashi, T.; Arai, S. Structure of (3-Chloro-2-hydroxy-5-nitrophenyl)-(2′-chlorophenyl)iodonium Hydroxide inner salt. J. Am. Chem. Soc. 1979, 101, 5858-5860.
34) Kita, Y.; Tohma, H.; Hatanaka, K.; Takada, T.; Fujita, S.; Mitoh, S.; Sakurai, H.; Oka, S. Hypervalent iodine-induced nucleophilic substitution of para-substituted phenol ethers. Generation of cation radicals as reactive intermediates. J. Am. Chem. Soc. 1997, 116, 3684-3691.
35) Doebner, O. Knoevenagel Condensation; Doebner Modification. Ber. 1900, 33, 2140.
36) Draper, R. W.; Radha, B. H.; Iyer, R. V.; Li, X.; Lu, Y.; Rahman, M.; Vater, E. J. An efficient process for the synthesis of trans-2.3-disubstituted-2,3-dihydro-4H-1-benzopyran-4-ones (Chroman-4-ones). Tetrahedron. 2000, 56, 1811-1817.
37) Smith, M. B.; March, J. March's Advanced Organic Chemistry Reactions, Mechanisms and Structure. John Wiley & Sons, Inc., Toronto, Canada, 2001, pp. 1544-1546.
38) Greene, T. W.; Wuts, P. G. M. Protective Groups in Organic Synthesis, 3^{rd} Edition, John Wiley & Sons Inc., Canada, 1999, p. 552.
39) Magdziak, D.; Meek, J. S.; Petus, T. R. R. Cyclohexadienone ketals and quinols: four building blocks potentially useful for enantioselective synthesis. Chem. Rev. 2004, 104, 1383-1429.
40) Marco-Contelles, J.; Teresa Molina, M.; Anjum, S, Naturally occurring cyclohexane epoxides: sources, biological activities, and synthesis. Chem. Rev. 2004, 104, 2857-2900.
41) Runcie, K. A.; Taylor, R. J. K. A short and efficient route to novel scyphostatin analogues. Organic Letters. 2001, 3 3237-3239.
42) Matsumoto, N.; Ariga, A.; To-e, S.; Nakamura, H.; Agata, N.; Hirano, S.; Inoue, J.; Umezawa, K. Synthesis of NF-κB activation inhibitors derived from epoxyquinomicin C. Bioorganic & Medicinal Chemistry Letters. 2000, 10, 865-869.
43) Barros, M. T.; Matias, P. M.; Maycock, C. D.; Ventura, M. R. Aziridines as a protecting and directing group. Stereoselective synthesis of (+)-bromoxone. Organic Letters. 2003, 5, 4321-4323.
44) Barrett, A. G. M.; Blaney, F.; Campbell, A. D.; Hamprecht, D.; Meyer, T.; White, A. J. P.; Witty, D., Williams, D. J. Unified route to the palmarumycin and preussomerin natural products. Enantioselective synthesis of (−)-preussomerin G. J. Org. Chem. 2002, 67, 2735-2750.
45) Nicolaou, K. C.; Sugita, K.; Baran, P. S.; Zhong, Y.-L. Iodine(V) reagents in organic synthesis. Part 2. Access to complex molecular architectures via Dess-Martin Periodinan-generated o-imidoquinones. J. Am. Chem. Soc. 2002, 124, 2221-2232.
46) Alcaraz, L.; Macdonald, G.; Ragot, J. P.; Lewis, N.; Taylor, R. J. K. Manumycin A: Synthesis of the (+)-enantiomer and revision of stereochemical assignment. J. Org. Chem. 1998, 63, 3526-3527.
47) Suzuki, Y.; Sugiyama, C.; Ohno, O.; Umezawa, K. Preparation and biological activities of optically active dehydromethylepoxyquinomicin, a novel NF-κB inhibitor. Tetrahedron. 2004, 60, 7061-7066.
48) Takagi, R.; Miyanaga, W.; Tamura, Y.; Kojima, S.; Ohkata, K. π-facial selectivity in Diels-Alder reactions of cross-conjugated ketones bearing an oxa-spiro-ring with sterically undemanding dienes. Heterocycles. 2003, 60, 785-790.
49) Ohkata, K.; Tamura, Y.; Shetuni, B. B.; Takagi, R.; Miyanaga, W.; Kohima, S.; Paquette, L. A. Stereoselectivity control by oxaspiro rings during Diels-Alder cycloadditions to cross-conjugated cyclohexadienones: The syn oxygen phenomenon. J. Am. Chem. Soc. 2004, 126, 16783-16792.
50) Takagi, R.; Miyanaga, W.; Tamura, Y.; Ohkata, K. Efficient synthesis of a 4,5-epoxy-2-cyclohexen-1-one derivative bearing a spirolactone via a Diels-Alder reaction with high α-facial selectivity: a synthetic study towards scyphostatin. Chem. Commun. 2002, 2096-2097.
51) Paquette, L. A.; Shetuni, B. B.; Gallucci, J. C. π-facial stereoselectivity in Diels-Alder cycloadditions to 1-oxaspiro[4,5]deca-6,9-dien-8-one. The strong directive effect of ether oxygen in a cross-conjugated ketone setting. Org. Lett. 2003, 5, 2639-2642.
52) Messere, A.; Gentili, A.; Garella, I.; Temussi, F.; Di Blasio, B.; Fiorentino, A. Nitration of cinnamic acids using cerium (IV) ammonium nitrate immobilized on silica. Syn. Comm. 2004, 34, 3317-3324.
53) Committee for Proprietary Medical Products. Working parties on quality, safety and efficacy of medical products. Note for guidance: investigation of chiral active substances. 1993, III/3501/91.
54) Rouhi, A. M. Moving beyond natural products. Chem. Eng. News, 2003, 81, 104-107.
55) Plourde, G. L.; Fisher, B. B. Synthesis of 6-methoxy-1-oxaspiro[4,5]deca-6,9-diene-8-one. Molecules. 2002, 7, 315-319.
56) Wipf, P.; Kim, Y. Stereoselective synthesis of the functionalized spirocyclic core of aranorosin. J. Org. Chem. 1993, 58, 1649-1650.
57) Tamura, Y.; Yakura, T.; Haruta, J, Kita, Y. Hypervalent iodine oxidation of p-alkoxyphenols and related compounds: A general route to p-benzoquinone monoacetals and spirolactones. J. Org. Chem. 1987, 52, 3927-3930.
58) Donohoe, T. J. Oxidation and Reduction in Organic Synthesis. Oxford University Press, New York, N.Y., 2000, pp. 13-16.
59) Suzuki, Y.; Sugiyama, C.; Ohno, O.; Umezawa, K. Preparation and biological activities of optically active dehydromethylepoxyquinomicin, a novel NF-κB inhibitor. Tetrahedron. 2004, 60, 7061-7066.
60) Danishefsky, S.; Zamboni, R.; Kahn, M.; Etheredge, S. J. Stereospecific total syntheses of dl-coriolin and dl-coriolin B. J. Am. Chem. Soc. 1981, 103, 3460-3467.
61) Matsumoto, N.; Ariga, A.; To-e, S.; Nakamura, H.; Agata, N.; Hirano, S.; Inoue, J.; Umezawa, K. Synthesis of NF-κB activation inhibitors derived from epoxyquinomicin C. Bio. Med. Chem. Lett. 2000, 10, 865-869.
62) Van Hijfte, L.; Little, R. D.; Petersen, J. L.; Moeller, K. D. Intramolecular 1,3-diyl trapping reactions. Total synthesis of (+/−)-hypnophilin and (+/−)-coriolin. Formation of the trans-fused bicyclo[3.3.0]octane ring system. Org. Chem. 1987, 52, 4647-4661.
63) Adam, W.; Rao, P. B.; Degen, H.; Levai, A.; Patonay, T.; Saha-Moller, C. R. Asymmetric Weitz-Scheffer epoxidation of isoflavones with dydroperoxides mediated by optically active phase transfer catalysts. J. Org. Chem. 2002, 67, 259-264.
64) Marco-Contelles, J.; Molina, M. T.; Anjum, S. Naturally occurring cyclohexane epoxides: Sources, biological activities and synthesis. Chem. Rev. 2002, 1-44.

### APPENDIX 1

A Table of Manumycin Structures^{53,54}

| | | |
|---|---|---|
| Compound | R₁ | R₂ |

Type I-a

| | | | |
|---|---|---|---|
| 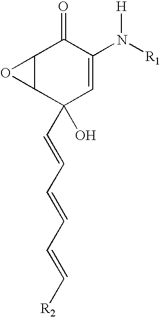 | Alisamycin | 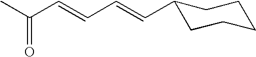 | 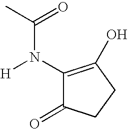 |
| | Ent-alisamycin | 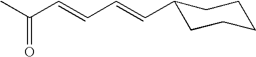 | |
| | Asukamycin | 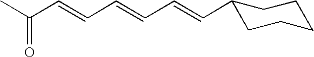 | |
| | Colaboymcin A | 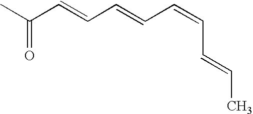 | 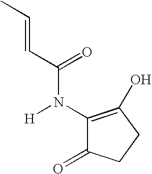 |
| | Compound 1 | 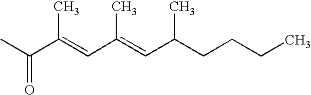 | CO₂H |
| | EI-1511-3 | 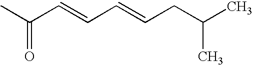 | 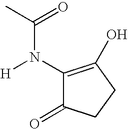 |
| | EI-1511-5 | 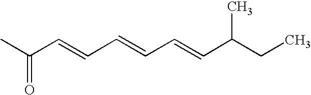 | |
| | EI-1625-2 | 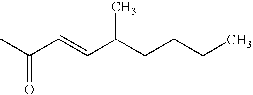 | |
| | Manumycin A | 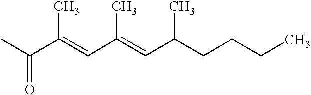 | |
| 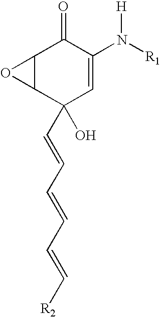 | Manumycin B | 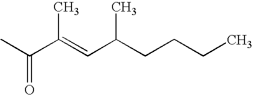 | 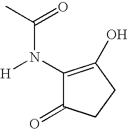 |
| | Manumycin C | 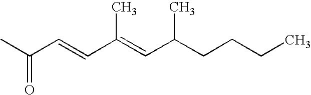 | |
| | Manumycin E | 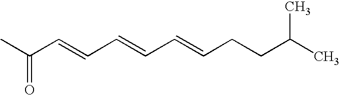 | |
| | Manumycin F | 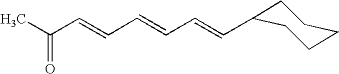 | |
| | Manumycin G | 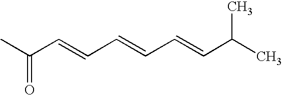 | |
| | Nisamycin | 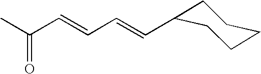 | CO₂H |
| | U-56, 407 | 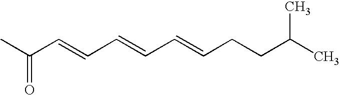 | |
| 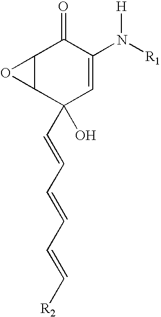 | Manumycin B | 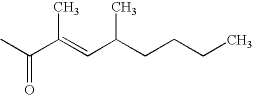 | 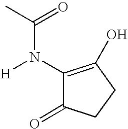 |
| | Manumycin C | 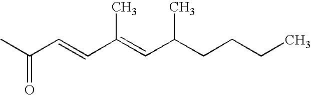 | |
| | Manumycin E | 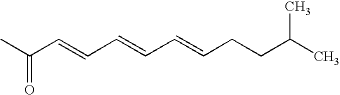 | |
| | Manumycin F | 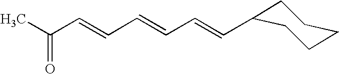 | |
| | Manumycin G | 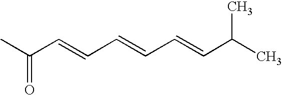 | |
| | Nisamycin | 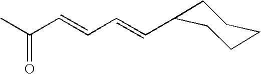 | CO₂H |
| | U-56, 407 | 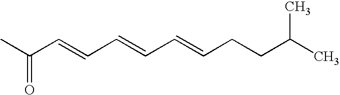 | |
| Type I-b | | | |
| 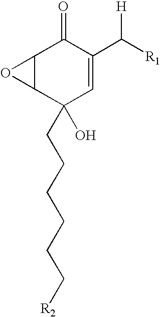 | U-62 162 | 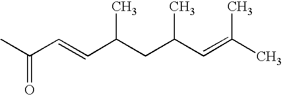 | CO₂H |
| Type II | | | |
| 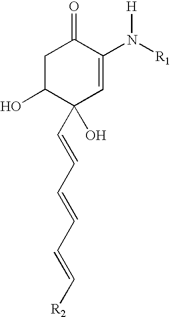 | Colabomycin D | 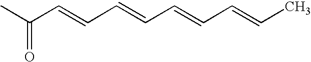 | 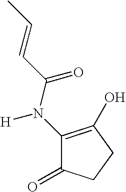 |
| | Manumycin D | 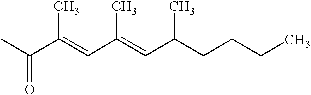 | 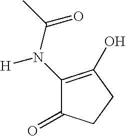 |
| | TMC-1A | 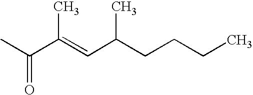 | |
| | TMC-1B | 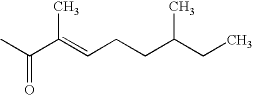 | |
| 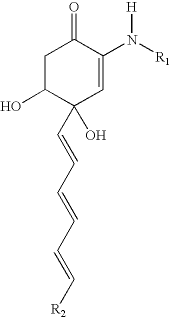 | TMC-1C | 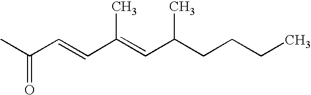 | 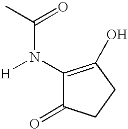 |
| | TMC-1D | 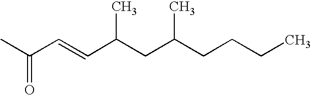 | |
| | Asukamycin A-II | 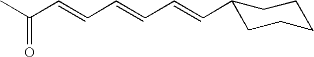 | |
| | Asukamycin B-II | 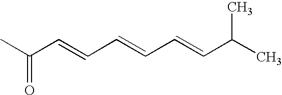 | |
| | Asukamycin C-II | 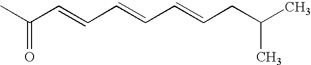 | |
| | Asukamycin D-II | 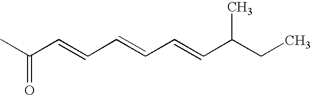 | |
| | Asukamycin E-II | 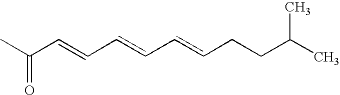 | |

### APPENDIX 2

### NMR Spectra of Compounds in Numerical Order (All Values are in ppm)

¹H-NMR Spectrum of Intermediate to Compound 28

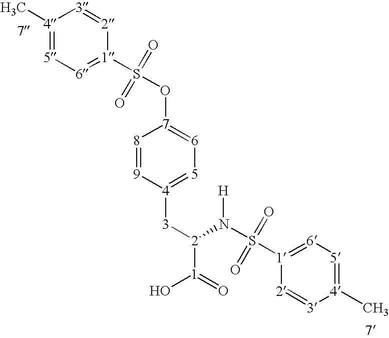

¹³C-NMR Spectrum of Intermediate to Compound 28

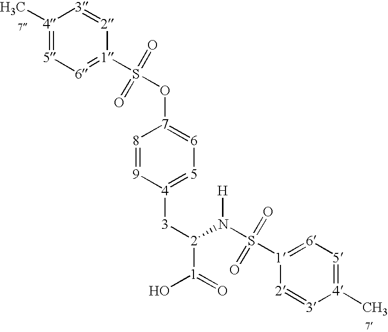

¹H-NMR Spectrum of Compound 28

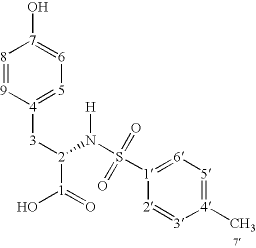

¹³C-NMR Spectrum of Compound 28

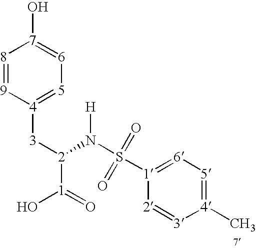

COSY-NMR Spectrum of Compound 28

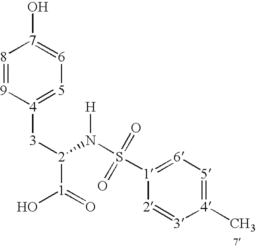

HETCOR-NMR Spectrum of Compound 28

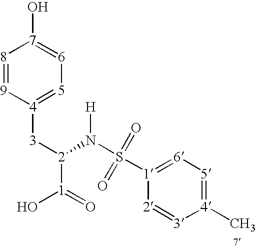

¹H-NMR Spectrum of Compound 29

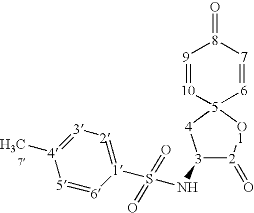

¹H-NMR Spectrum of Compound 30

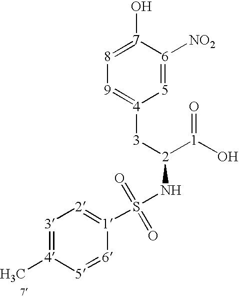

¹³C-NMR Spectrum of Compound 30

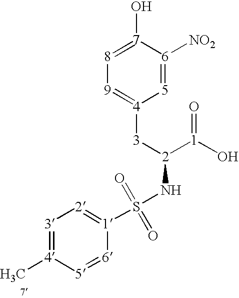

COSY-NMR Spectrum of Compound 30

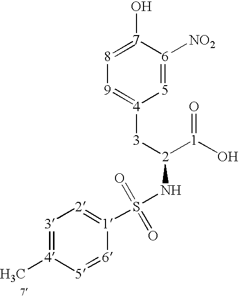

COSY-NMR Spectrum of Compound 30

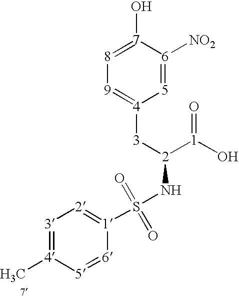

HETCOR-NMR Spectrum of Compound 30

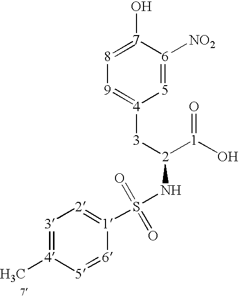

¹H-NMR Spectrum of Compound 32

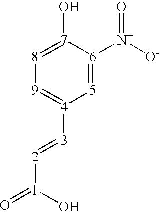

¹H-NMR Spectrum of Crude Compound 33

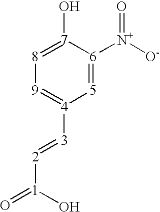

¹H-NMR Spectrum of Compound 34

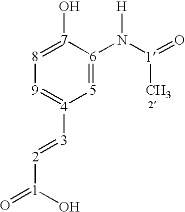

¹³C-NMR Spectrum of Compound 34

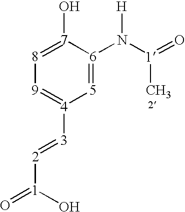

¹H-NMR Spectrum of Compound 35

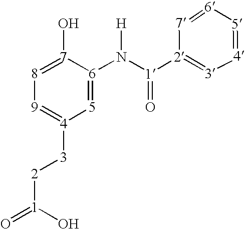

¹³C-NMR Spectrum of Compound 35

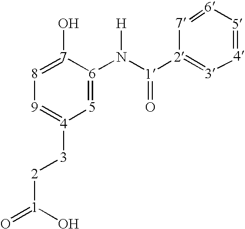

¹³C-NMR Spectrum of Compound 35

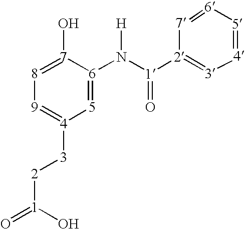

COSY-NMR Spectrum of Compound 35

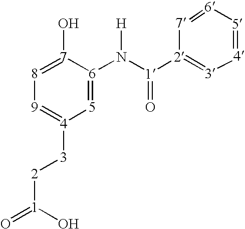

COSY-NMR Spectrum of Compound 35

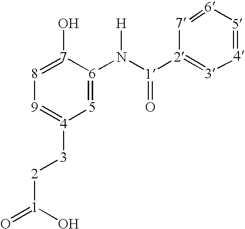

HETCOR-NMR Spectrum of Compound 35

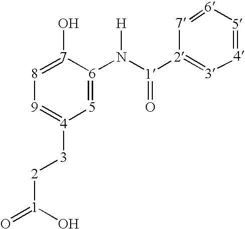

¹H-NMR Spectrum of Compound (+/−)-36

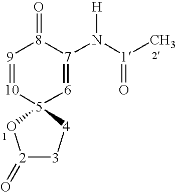

¹³C-NMR Spectrum of Compound (+/−)-36

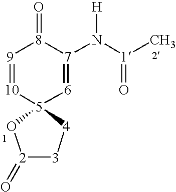

¹H-NMR Spectrum of Compound (+/−)-37

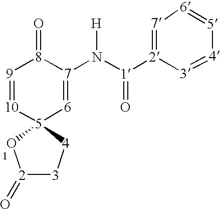

¹³C-NMR Spectrum of Compound (+/−)-37

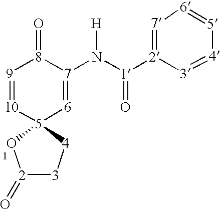

¹H-NMR Spectrum of Compound (+/−)-39

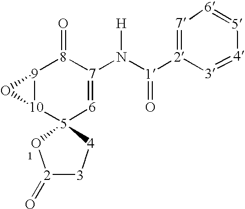

¹H-NMR Spectrum of Compound (+/−)-39

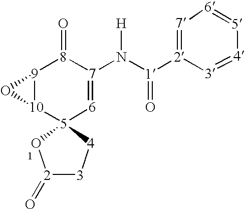

¹³C-NMR Spectrum of Compound (+/−)-39

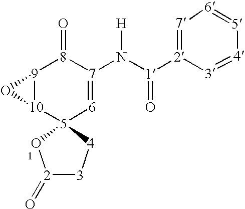

DEPT-135-NMR Spectrum of Compound (+/−)-39

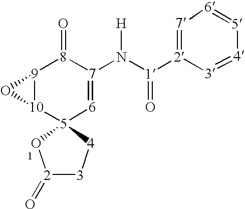

¹H-NMR Spectrum of Compound 40

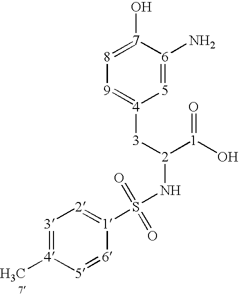

¹H-NMR Spectrum of Compound 41

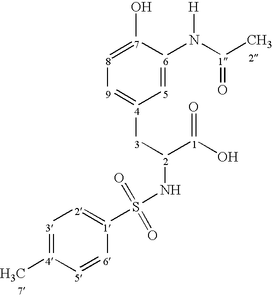

¹³C-NMR Spectrum of Compound 41

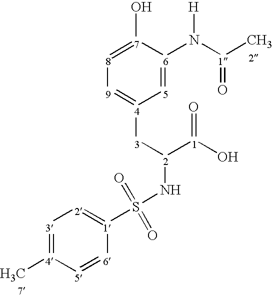

¹H-NMR Spectrum of Compound (+)-42 Major Diastereomer

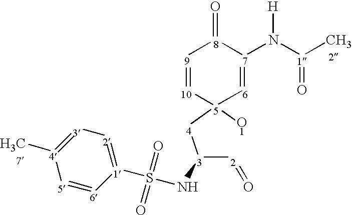

¹³C-NMR Spectrum of Compound (+)-42 Major Diastereomer

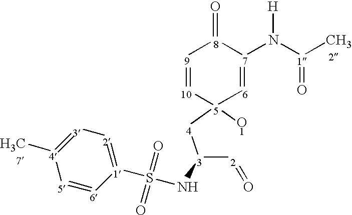

DEPT-NMR Spectrum of Compound (+)-42, Major Diastereomer

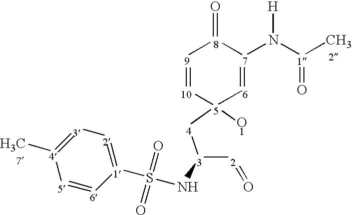

HETCOR-NMR Spectrum of Compound (+)-42, Major Diastereomer

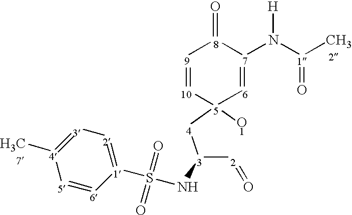

HETCOR-NMR Spectrum of Compound (+)-42 Major Diastereomer

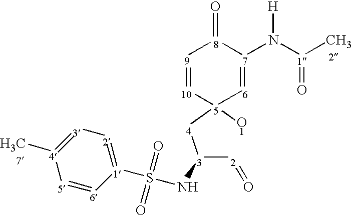

¹H-NMR Spectrum of Crude Product 42, Diastereomers

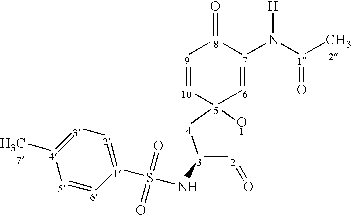

¹H-NMR Spectrum of Crude Product 42, Diastereomers

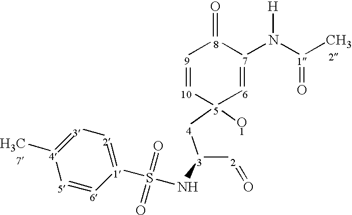

¹H-NM Spectrum of Crude Product 42, Diastereomers

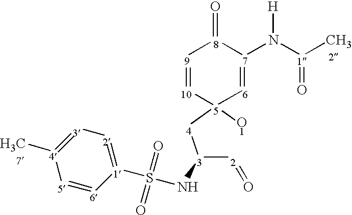

¹H-NMR Spectrum of Crude Product 42, Diastereomers

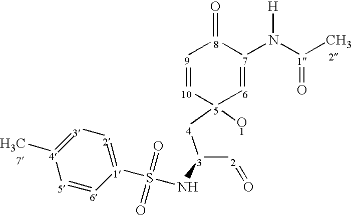

¹H-NMR Spectrum of Crude Product 42, Diastereomers

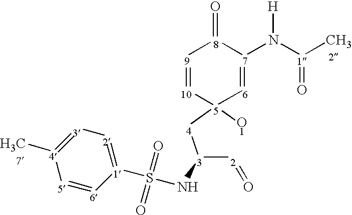

What is claimed is:

1. A compound having the chemical formula (I): 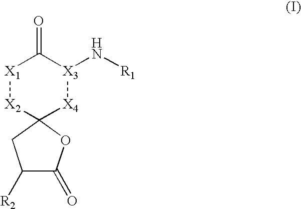
wherein X₁ and X₂ are carbon atoms either joined by double bond or joined by a single bond and comprising constituents of an epoxide ring or a hydroxyethylene moiety;
wherein X₃ and X₄ are carbon atoms either joined by double bond or joined by a single bond and comprising constituents of an epoxide ring;
wherein R₁ is selected from the group consisting of branched alkyl chains, unbranched alkyl chains, cycloalkyl groups, aromatic groups, alcohols, ethers, amines, and substituted or unsubstituted ureas, esters, aldehydes and carboxylic acids; and
wherein R₂ is selected from the group consisting of H, OH and NHR₃ wherein R₃ is a nitrogen protecting group.

2. The compound as defined in claim 1, wherein said compound is an optically pure stereoisomer.

3. The compound as defined in claim 1, wherein said compound is a diastereomer.

4. The compound as defined in claim 1, wherein said compound is a racemate.

5. The compound as defined in compound 1, wherein R₁ comprises a polyunsaturated carbon chain having between 2 and 12 carbon atoms.

6. The compound as defined in claim 1, wherein R₁ is selected from the group consisting of the following substituents:

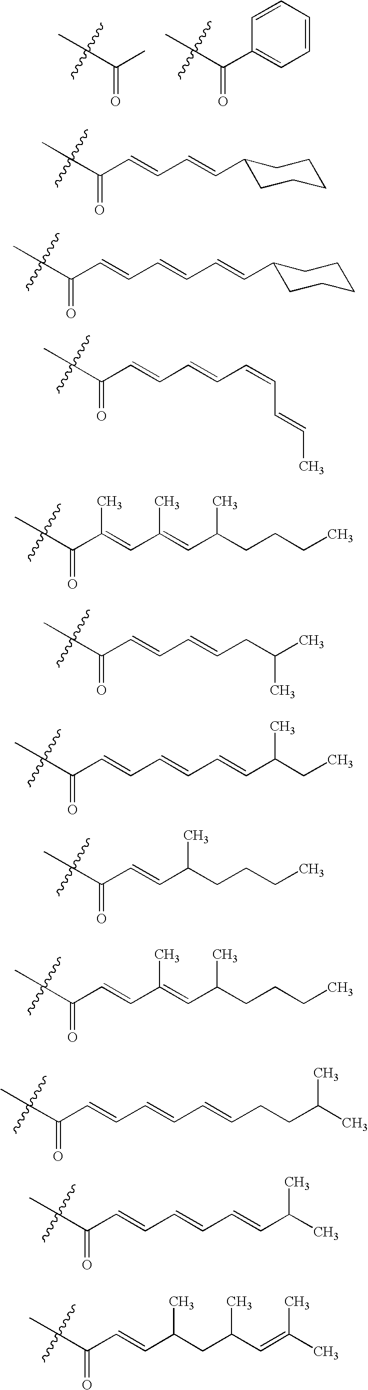

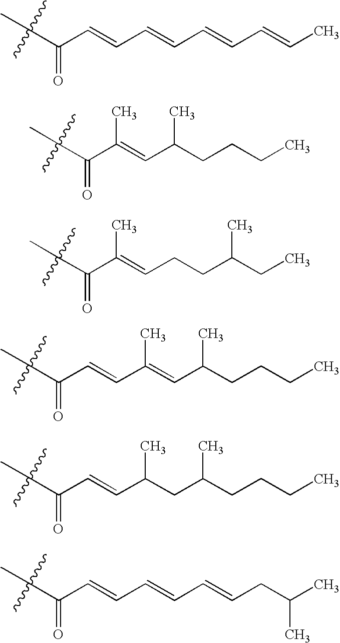

7. The compound as defined in claim 1, wherein said compound is an analogue of a naturally occurring compound selected from the group consisting of aranorosins, gymnastatins and manumycins.

8. The compound as defined in claim 1, wherein R₃ is a tosyl group.

9. The compound as defined in claim 71, wherein said R₃ is a sulphonyl chain selected from the group consisting of the following substituents:

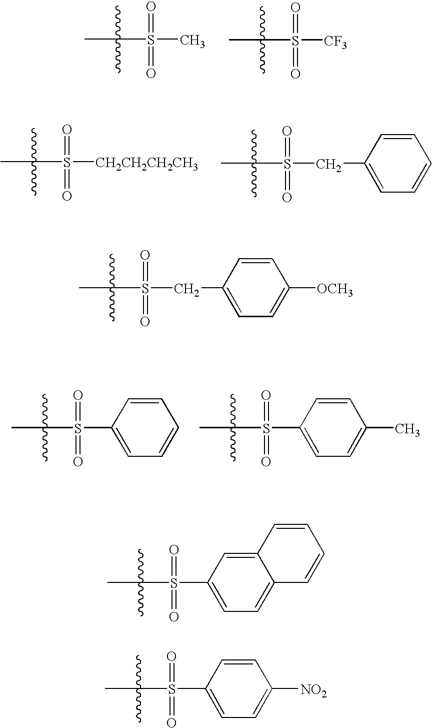

10. The compound as defined in claim 1, wherein R₃ comprises a 4,6-dimethyl-dodecadiene-2E,4E-oic acid moiety.

11. The compound as defined in claim 1, wherein X₁ and X₂ are constituents of an epoxide ring and X₃ and X₄ are joined by a double bond.

12. A pharmaceutical composition comprising an effective amount of the compound defined in claim 2 together with a pharmaceutically acceptable carrier.

13. A pharmaceutical composition comprising an effective amount of the compound defined in claim 6 together with a pharmaceutically acceptable carrier.

14. A pharmaceutical composition comprising an effective amount of the compound defined in claim 7 together with a pharmaceutically acceptable carrier.

15. A method of diasteroselective formation of a spirolactone comprising:
(a) providing a starting material selected from the group consisting of tyrosine or a tyrosine derivative, wherein said starting material comprises a tethered chiral chain comprising an amino functional group;
(b) selectively protecting said amino functional group to produce an amino protected intermediate; and
(c) oxidatively spiroannulating said protected intermediate or a derivative thereof to preferentially form a diastereomer of said spirolactone.

16. The method as defined in claim 15, wherein said spiroannulation produces a mixture of diastereomers in a non-equal ratio and wherein said method comprises separating said diastereomers.

17. The method as defined in claim 16, wherein diastereomers are separated by chromatography.

18. The method as defined in claim 15, wherein said selectively protecting said amino functional group comprises tosylating said starting material.

19. The method as defined in claim 15, wherein said selectively protecting said amino functional group comprises reacting said starting material with a sulphonyl chloride selected from the group consisting of the following compounds:

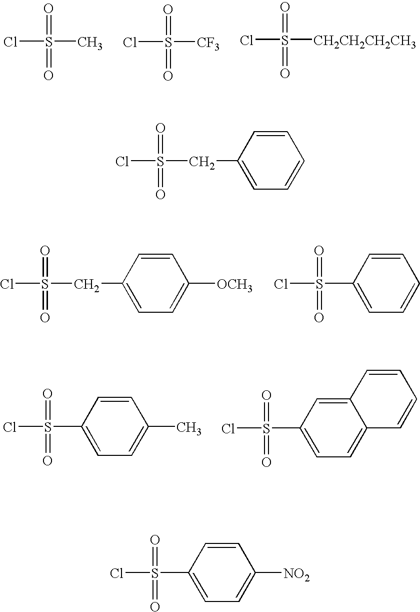

20. The method as defined in claim 15, comprising epoxidating said spirolactone to form an oxirane spirolactone derivative.

21. The method as defined in claim 15, wherein said starting material is selected from the group consisting of (L)-3-nitro-tyrosine and (D)-3-nitro-tyrosine.

22. The method as defined in claim 21, comprising hydrogenating said starting material or a derivative thereof to transform said nitro group to an amine group and thereby produce an electron donating amine derivative.

23. The method as defined in claim 22, comprising, prior to said spiroannulation, reacting said amine derivative with an acid chloride derivative selected from the group consisting of the following compounds to produce an amide derivative:

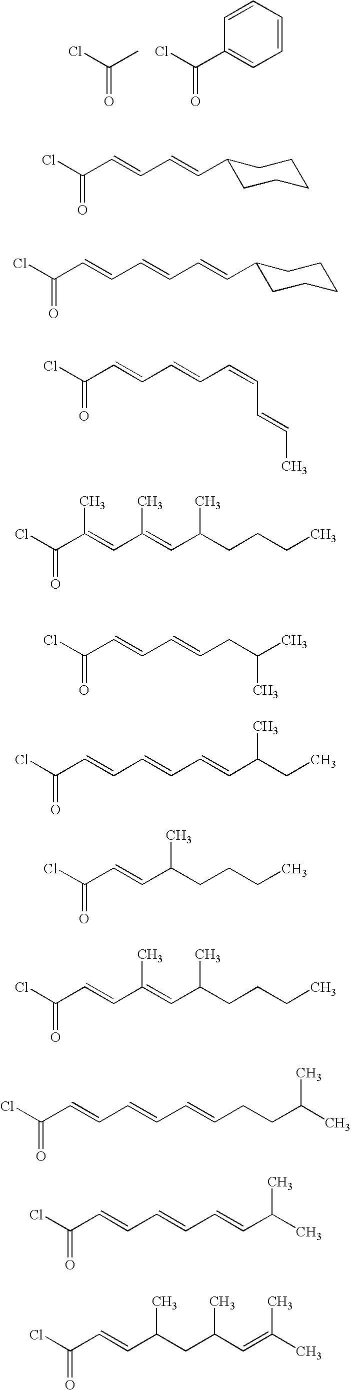

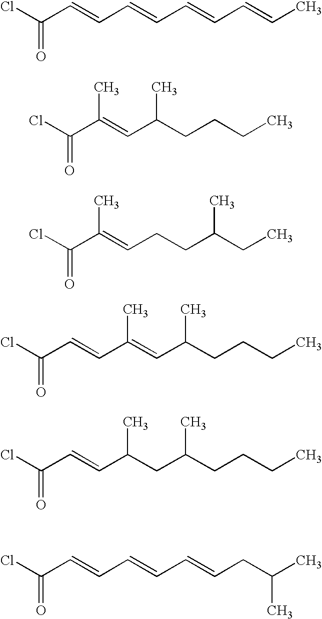

24. The method as defined in claim 20, wherein said epoxidating comprises reacting said spirolactone with an oxidizing agent, wherein said oxidizing agent preferentially attacks either an electron rich double bond or an electron poor double bond, or both double bonds, of said spirolactone.

25. A method of forming a para-quinol derivative comprising:
(a) providing a phenol starting material comprising a nitro functional group;
(b) hydrogenating said starting material or a derivative thereof to transform said nitro group to an amine group and thereby produce an electron donating amine derivative;
(c) reacting said amine derivative with an acid chloride derivative to produce an amide derivative; and
(d) oxidatively spiroannulating said amide derivative to produce a spirolactone.

26. The method as defined in claim 25, wherein said spiroannulating is diastereoselective.

27. The method as defined in claim 25, comprising epoxidating said spirolactone to produce an oxirane derivative.

28. The method as defined in claim 25, comprising cleaving the lactone ring of said oxirane derivative to form a manumycin analogue.

29. The method as defined in claim 25, wherein said starting material comprises a chiral centre.

30. The method as defined in claim 25, wherein said starting material is selected from the group consisting of 4-hydroxy-3-nitrobenzaldehyde, tyrosine and 3-nitro-tyrosine.

31. The method as defined in claim 30, wherein said acid chloride derivative is selected from the group consisting of the following compounds:

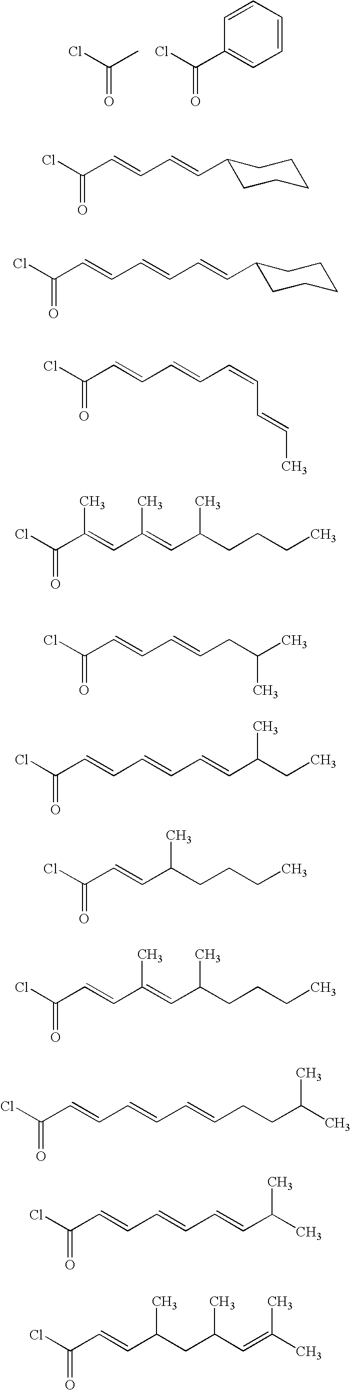

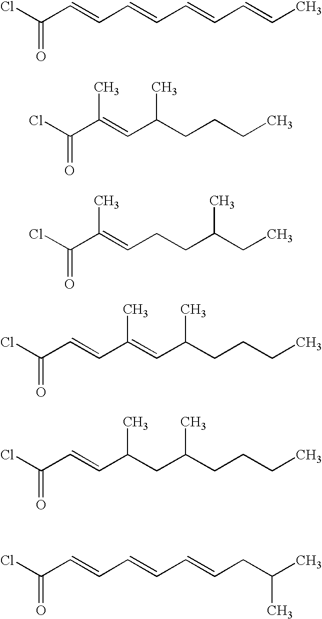

32. The method as defined in claim 30, wherein said starting material comprises an amino moiety and wherein said method comprises reacting said starting material with a nitrogen protecting group to functionalize said amino moiety prior to said hydrogenating.

33. The method as defined in claim 30, wherein said nitrogen protecting group is sulphonyl chloride selected from the group consisting of the following compounds:

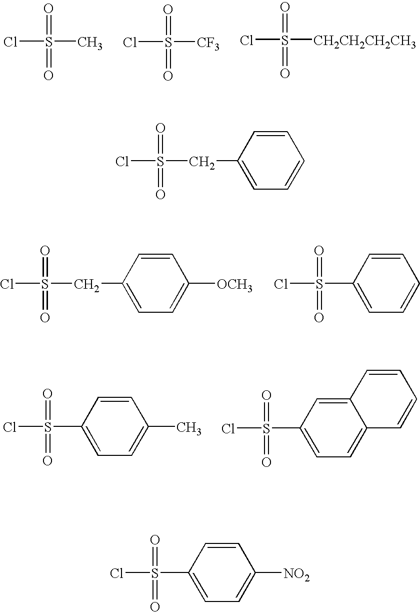

34. The method as defined in claim 25, comprising the synthetic steps set forth in Scheme 7:

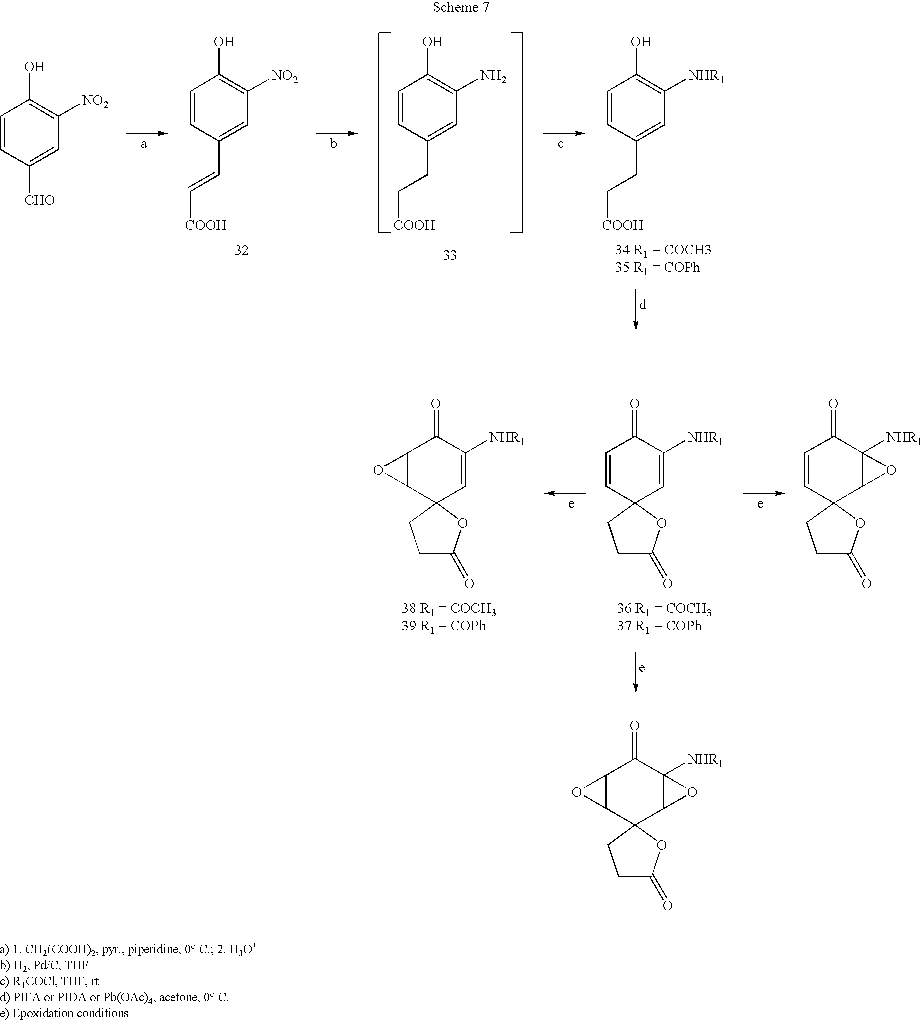

35. The method as defined in claims 15 or 25, comprising the synthetic steps set forth in Scheme 8:

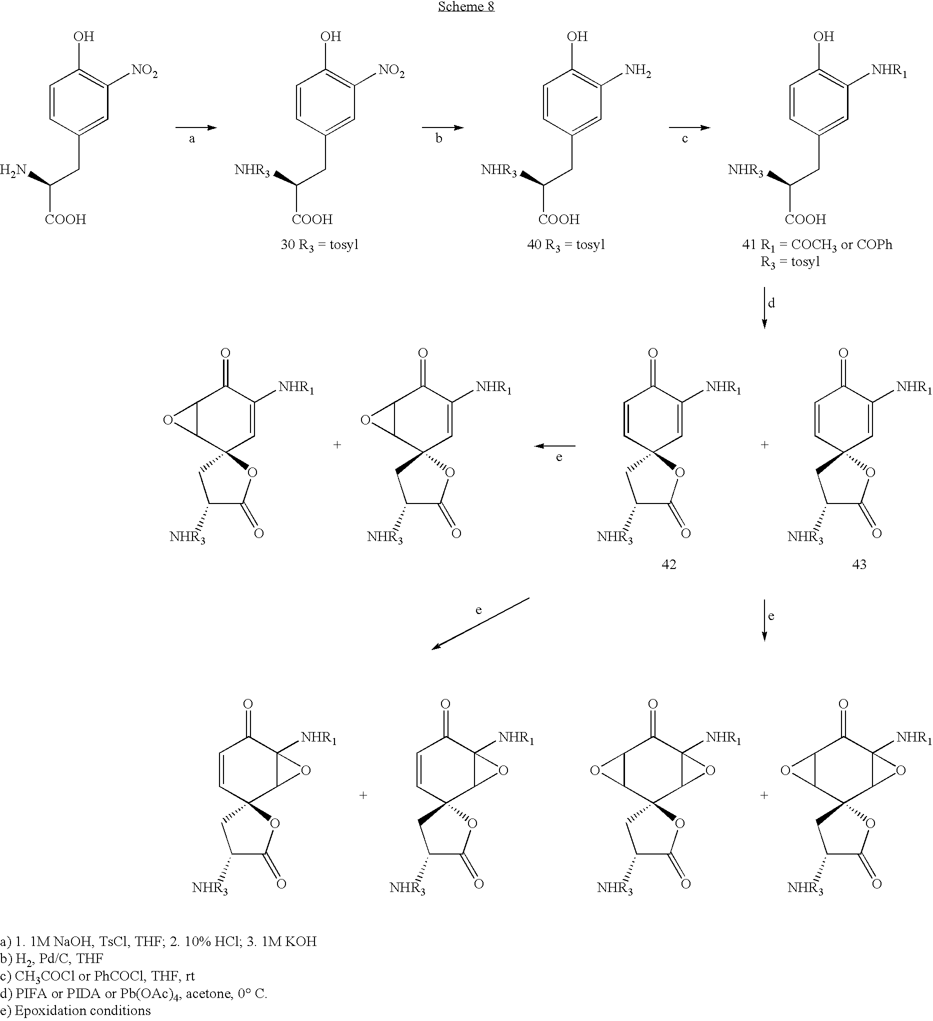

36. A diasteroselective method of synthesizing a compound (I) as defined in claim 1 comprising the steps of:
(a) providing a nitrotyrosine compound having an amino moiety having a chiral centre;
(b) protecting said amino moiety of said nitrotyrosine compound to produce a protected derivative;
(c) hydrogenating said protected derivative to produce an amine derivative having a protected amine group and an unprotected amine group;
(d) reacting said amine derivative with an acid chloride derivative to produce an amide derivative; and
(e) oxidatively spiroannulating said amide derivative to produce said compound.

37. The method as defined in claim 36, comprising separating diastereoisomers of said compound to yield an optically pure stereoisomer.

38. The method as defined in claim 36, further comprising subjecting the product of said spiroannulating to expoxidation.

39. The use of the compound as defined in claim 1 as an antibiotic, antiviral, antifungal, anti-inflammatory, antiparasitic or anti-cancer therapeutic agent.

40. The use of the composition as defined in claim 12 an antibiotic, antiviral, antifungal, anti-inflammatory, antiparasitic or anti-cancer therapeutic agent.

41. A salt or prodrug of the compound as defined in claim 1.

42. The compound as defined in claim 1, wherein compound (I) is selected from the group consisting of: [chemical structure]
